(19) United States
(12) Patent Application Publication
(10) Pub. No.: US 2021/0393618 A1
(43) Pub. Date: Dec. 23, 2021

(54) BUPRENORPHINE TO TREAT RESPIRATORY DEPRESSION

(71) Applicant: INDIVIOR UK LIMITED, Hull (GB)

(72) Inventors: Robert DOBBINS, North Chesterfield, VA (US); Anne C. ANDORN, North Chesterfield, VA (US); Frank GRAY, North Chesterfield, VA (US); Susan LEARNED, North Chesterfield, VA (US); Christian A. HEIDBREDER, North Chesterfield, VA (US)

(21) Appl. No.: 17/283,931

(22) PCT Filed: Oct. 11, 2019
(86) PCT No.: PCT/US2019/055890

§ 371 Date: Apr. 8, 2021

Related U.S. Application Data
(60) Provisional application No. 62/817,136, filed on Mar. 12, 2019.
(60) Provisional application No. 62/795,258, filed on Jan. 22, 2019.
(60) Provisional application No. 62/744,530, filed on Oct. 11, 2018.

(51) Int. Cl. A61K 31/485; A61K 47/32; A61K 47/34; A61K 9/00; A61P 11/00

(52) U.S. Cl. CPC A61K 31/485; A61K 47/32; A61P 11/00; A61K 9/0019; A61K 47/34

(57) ABSTRACT

The disclosure provides methods for treating, preventing, and reducing the incidence of opioid-induced respiratory depression, opioid-induced apnea, and opioid overdose in patients by administering buprenorphine or a pharmaceutically acceptable salt thereof.

### CROSS-REFERENCE TO RELATED APPLICATIONS

This application claims priority to U.S. Application No. 62/817,136 filed Mar. 12, 2019; U.S. Application No. 62/795,258 filed Jan. 22, 2019; and U.S. Application No. 62/744,530 filed Oct. 11, 2018, the disclosures of which are incorporated by reference herein in their entirety.

### BACKGROUND

Prescription opioid medications, such as oxycodone, are amongst the most commonly used analgesics. Physicians worldwide prescribe opioids for patients in many different clinical settings, including patients with post-operative pain or pain due to cancer. Unfortunately, opioid medications can also be diverted or illegally synthesized and used in an illicit manner to produce euphoria. Administration of prescription or illegal opioids can result in many unwanted pharmacological effects, including opioid-induced respiratory depression, which is potentially fatal. Opioids activate μ-opioid receptors at specific sites in the central nervous system (CNS) including the pre-Bötzinger complex, a respiratory rhythm generating area in the pons, causing decreased ventilatory response. The result can be hypercapnia, hypoxia and irregular breathing, and at high dosages, a complete cessation of rhythmic respiratory activity.

In patients with opioid use disorder, marked by excessive or uncontrolled use of opioids, respiratory depression is a major cause of death. According to the 2016 National Survey on Drug Use and Health, there were 20.1 million people aged 12 or older in the Unites States of America (USA) who suffered from substance use disorder. In 2015 alone, the harm caused by drug use translated into an estimated 28 million years of healthy life lost worldwide due to premature death and disability. In the USA, the misuse of opioids accounted for approximately 25% of the number of drug-related deaths worldwide, increasing from 16,849 to 52,404 annually during the 1999-2015 period. In Europe, the use of prescription opioids has also increased, but this change has not been associated with a marked increase in overdose deaths. Still, some regions have reported dramatic increases in deaths due to overdose, and the common thread among these reports has been widespread access to highly potent fentanyl and carfentanil. The risk of overdose deaths is particularly high for problem drug users after periods of relative abstinence, most notably soon after prison release, but also after hospital discharge. The most probable reason is loss of tolerance to the effects of opioids, but the changes in tolerance have not been well-characterized.

There is a need in the art to shift the respiratory depression response to intravenous opioid injections to the right, thereby inhibiting the actions of more potent opioids, such as fentanyl, in causing respiratory depression—the usual fatal precipitant associated with intravenous opioid overdose. The disclosure is directed to this, as well as other, important ends.

### SUMMARY

The disclosure provides methods of treating or preventing fentanyl-induced respiratory depression in a patient in need thereof by administering to the patient a therapeutically effective amount of a pharmaceutical composition comprising buprenorphine free base, a poly(lactide-co-glycolide) copolymer, and N-methyl-2-pyrrolidone, to treat or prevent fentanyl-induced respiratory depression.

The disclosure provides methods of reducing the incidence of fentanyl-induced respiratory depression in a patient in need thereof by administering to the patient a therapeutically effective amount of a pharmaceutical composition comprising buprenorphine free base, a poly(lactide-co-glycolide) copolymer, and N-methyl-2-pyrrolidone, to reduce the incidence of fentanyl-induced respiratory depression.

The disclosure provides methods treating or preventing opioid-induced respiratory depression or reducing the incidence of opioid-induced respiratory depression in a patient in need thereof by administering to the patient a therapeutically effective amount of a pharmaceutical composition comprising buprenorphine or a pharmaceutically acceptable salt thereof. In aspects, the opioid is fentanyl, a fentanyl analogue, carfentanil, or a carfentanil analogue.

The disclosure provides methods of treating or preventing opioid-induced apnea, reducing the incidence of opioid-induced apnea, treating or preventing an opioid overdose, or reducing the incidence of opioid overdose in a patient in need thereof, the method comprising administering to the patient a therapeutically effective amount of a pharmaceutical composition comprising buprenorphine or a pharmaceutically acceptable salt thereof. In aspects, the opioid is fentanyl, a fentanyl analogue, carfentanil, or a carfentanil analogue.

These and other aspects and embodiments of the disclosure are described in more detail herein.

### BRIEF DESCRIPTION OF THE FIGURES

FIG. 1 is an overview of the clinical study design in healthy subjects, as described in the example. “BUP” refers to buprenorphine.

FIGS. 2A-2B are an overview of the clinical study design in opioid-tolerant patients, as described in Example 2.

FIG. 3 shows arterial plasma buprenorphine concentrations for patients dosed with buprenorphine at 0.1 mg/70 kg/hour (i.e., target dose of 1 ng/mL)(bottom line); 0.2 mg/70 kg/hour (i.e., target dose of 2 ng/mL)(middle line); and 0.5 mg/70 kg/hour (i.e., target dose of 5 ng/mL) (top line).

FIGS. 4A-4B show the end-tidal CO₂, minute ventilation and oxygen saturation (SpO₂) for the first patient to receive a low dose buprenorphine (i.e., 1 ng/mL) with fentanyl boluses, where FIG. 4A is placebo administration and FIG. 4B is administration of 1 ng/mL buprenorphine.

FIGS. 5A-5B show the end-tidal CO₂, minute ventilation and oxygen saturation (SpO₂) for the first patient to receive a middle dose buprenorphine (i.e., 2 ng/mL) with fentanyl boluses, where FIG. 5A is placebo administration and FIG. 5B is administration of 2 ng/mL buprenorphine.

FIGS. 6A-6B show the end-tidal CO₂, minute ventilation and oxygen saturation (SpO₂) for the first patient to receive a high dose buprenorphine (i.e., 5 ng/mL) with fentanyl boluses, where FIG. 6A is placebo administration and FIG. 6B is administration of 5 ng/mL buprenorphine.

### DETAILED DESCRIPTION

Many patients are not aware that addiction is a disease that can be medically treated. The goals of medication-assisted treatment are to reduce substance use and risk of relapse or overdose, to reduce harm from sequelae of substance abuse and to help patients return to a healthy, functional life. Buprenorphine, a partial agonist at the μ-opioid receptor is used for the medication-assisted treatment of opioid use disorder. Buprenorphine has high affinity for the μ-opioid receptor and therapeutic plasma concentrations achieve ≥70% receptor occupancy. As a partial agonist, buprenorphine has a ceiling effect on respiratory depression such that it does not cause apnea when administered alone and minute ventilation is not suppressed beyond 50 to 60%. The disclosure is directed to the surprising discovery that buprenorphine will competitively inhibit the effects of potent, short-acting μ-opioid receptor agonists, like fentanyl and carfentanil, that can result in respiratory depression, apnea and death.

Buprenorphine is a partial agonist at the μ-opioid receptor and an antagonist at the κ (kappa) receptor. Buprenorphine is oxidatively metabolized by 14-N-dealkylation to N-dealkyl-buprenorphine (also known as norbuprenorphine) via cytochrome P450 CYP3A4 and by glucuronidation of the parent molecule and the dealkylated metabolite. Norbuprenorphine is a p. agonist with weak intrinsic activity. Elimination of buprenorphine is bi- or tri-exponential, with a long terminal elimination phase of 20 to 25 hours, due in part to reabsorption of buprenorphine after intestinal hydrolysis of the conjugated derivative, and in part to the highly lipophilic nature of the molecule. Buprenorphine is essentially eliminated in the feces by biliary excretion of the glucuronidated metabolites (80%), the rest being eliminated in the urine.

“Opioid” refers to any opioid known in the art other than buprenorphine or a pharmaceutically acceptable salt thereof. In aspects, the opioid is μ-opioid receptor agonist other than buprenorphine or a pharmaceutically acceptable salt thereof. In other words, as used herein, the term “opioid” excludes buprenorphine or a pharmaceutically acceptable salt thereof. In aspects, the opioid is an opioid that is abused or misused by a patient. In aspects, the opioid is an opioid that is administered to a patient in a hospital, clinic, or doctor's office. In aspects, the opioid is an opioid that is prescribed to a patient by a physician or other health care professional. In aspects, the opioid is heroin. In aspects, the opioid is a prescription drug. In aspects, the opioid is fentanyl, carfentanil, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or an analog or derivative of one or more of the foregoing. In aspects, the opioid is fentanyl. In aspects, the opioid is carfentanil. In aspects, the opioid is a fentanyl analogue. In aspects, the opioid is a carfentanil analogue. In aspects, the opioid is heroin.

“Analog,” or “analogue” is used in accordance with its plain ordinary meaning within Chemistry and Biology and refers to a chemical compound that is structurally similar to another compound (i.e., a “reference” compound, such as fentanyl or carfentanil) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance to a reference compound.

Fentanyl is a potent opioid analgesic with a high affinity for the μ-opioid receptor. It is used as an analgesic supplement to general anesthesia or as the sole anesthetic. The onset of action is rapid. However, the maximum analgesic and respiratory depressant effect may not be noted for several minutes. The usual duration of action of the analgesic effect is approximately 30 minutes after a single intravenous dose of up to 100 μg. The potency of analgesia is dose-related and can be adjusted based on the pain level of the surgical procedure. Fentanyl is a lipid-soluble drug and its pharmacokinetics can be described in terms of a 3-compartment model. Following intravenous injection, there is a short distribution phase during which high concentrations of fentanyl are achieved quickly in well-perfused tissues such as the lungs, kidneys and brain. The drug is redistributed to other tissues; it accumulates more slowly in skeletal muscle and yet more slowly in fat, from which it is gradually released into the blood. Up to 80% of fentanyl is bound to plasma proteins. Fentanyl is primarily metabolized in the liver, and it is excreted mainly in the urine with less than 10% representing the unchanged drug. The terminal half-life of fentanyl is 3.7 hours.

“Fentanyl analogue” refers to an analogue of fentanyl. In aspects, a fentanyl analogue is a compound that exhibits mu-opioid receptor binding the same as fentanyl or greater than fentanyl or that exhibits mu-opioid receptor binding in an amount of about 0-25% less than fentanyl, or about 0-10% less than fentanyl, based on a standard in vitro or in vivo mu-opioid receptor binding assay. Exemplary fentanyl analogues include acetylfentanyl, butyrfentanyl, para-tolylfentanyl, 3-methylfentanyl, α-methylfentanyl, mefentanyl, phenaridine, ohmefentanyl, mirfentanil, and the like. In aspects, the fentanyl analogue is acetylfentanyl. In aspects, the fentanyl analogue is butyrfentanyl. In aspects, the fentanyl analogue is para-tolylfentanyl. In aspects, the fentanyl analogue is 3-methylfentanyl. In aspects, the fentanyl analogue is α-methylfentanyl. In aspects, the fentanyl analogue is mefentanyl. In aspects, the fentanyl analogue is phenaridine. In aspects, the fentanyl analogue is ohmefentanyl. In aspects, the fentanyl analogue is mirfentanil.

“Carfentanil analogue” refers to an analogue of carfentanil. In aspects, a carfentanil analogue is a compound that exhibits mu-opioid receptor binding the same as or greater than carfentanil or that exhibits mu-opioid receptor binding in an amount of about 0-25% less than fentanyl, or about 0-10% less than carfentanil, based on a standard in vitro or in vivo mu-opioid receptor binding assay. Exemplary carfentanil analogues include sufentanil, remifentanil, alfentanil, lofentanil, brifentanil, trefentanil, and the like. In aspects, the carfentanil analogue is sufentanil. In aspects, the carfentanil analogue is remifentanil. In aspects, the carfentanil analogue is alfentanil. In aspects, the carfentanil analogue is lofentanil. In aspects, the carfentanil analogue is brifentanil. In aspects, the carfentanil analogue is trefentanil.

“Respiratory depression” refers to slow and shallow breathing, which can lead to apnea or respiratory failure. In aspects, respiratory depression refers to breathing rates of less than 12 breaths per minute. Opioids are known to cause respiratory depression. See Dahan et al, Anesthesiology, 112:226-238 (2010).

“Apnea” or “apnoea” refer to the temporary cessation of breathing.

“Respiratory failure” refers to the complete cessation of breathing.

“Opioid overdose” is defined by the World Health Organization as having the following three signs and symptoms: (i) pinpoint pupils, (ii) unconsciousness, and (iii) respiratory depression.

“Opioid use disorder” is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition, as further described in the Diagnostic and Statistical Manual for Mental Disorders, 5th Edition, American Psychiatric Association, 2013 (also referred to herein as DSM5). In aspects, the opioid use disorder is moderate opioid use disorder. “Moderate opioid use disorder” is defined by reference to the DSM5 Opioid Use Disorder Checklist (ICD-9-CM code 304.00 or ICD-10-CM code F11.20) as having the presence of 4 or 5 symptoms indicated in the DSM5 Opioid Use Disorder Checklist. In aspects, the opioid use disorder is severe opioid use disorder. “Severe opioid use disorder” is defined by reference to the DSM5 Opioid Use Disorder Checklist (ICD-9-CM code 304.00 or ICD-10-CM code F11.20) as having the presence of 6 or more symptoms indicated in the DSM5 Opioid Use Disorder Checklist. In aspects, the opioid use disorder is moderate-to-severe opioid use disorder. Moderate-to-severe opioid use disorder refers to the presence of 4 or more symptoms indicated in the DSM5 Opioid Use Disorder Checklist. In aspects, the opioid use disorder is mild opioid use disorder. “Mild opioid use disorder” is defined by reference to the DSM5 Opioid Use Disorder Checklist (ICD-9-CM code 305.50 or ICD-10-CM code F11.10) as having the presence of 2 or 3 symptoms indicated in the DSM5 Opioid Use Disorder Checklist. In aspects, the opioid use disorder is mild-to-moderate opioid use disorder. Mild-to-moderate opioid use disorder refers to the presence of 2 to 5 symptoms indicated in the DSM5 Opioid Use Disorder Checklist. In aspects, “treating opioid use disorder” encompasses one or more of: (i) reducing opioid withdrawal symptoms, (ii) eliminating opioid withdrawal symptoms, (iii) reducing opioid craving, (iv) eliminating opioid craving, (v) reducing illicit opioid use, (vi) eliminating illicit opioid use, and (vii) inducing opioid abstinence.

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease. In aspects, treating refers to reducing the impact of an opioid overdose. In aspects, treating refers to reducing the extent or degree of a patient's respiratory depression, e.g., in comparison to the extent or degree of the respiratory depression that would have occurred absent the buprenorphine or the pharmaceutically acceptable salt thereof. In aspects, treating refers to reducing the extent or degree of a patient's apnea, e.g., in comparison to the extent or degree of the apnea that would have occurred absent the buprenorphine or the pharmaceutically acceptable salt thereof. Treatment methods include administering to a subject a therapeutically effective amount of buprenorphine or a pharmaceutically acceptable salt thereof. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment may increase or decrease over the course of a particular treatment. Changes in dosage may result and become apparent by standard diagnostic assays known in the art. In some instances, buprenorphine is administered to the subject in an amount and for a duration sufficient to treat the patient.

“Preventing” refers to preventing the occurrence of respiratory depression, apnea, or an opioid overdose in a patient that would have occurred absent the presence of the buprenorphine or pharmaceutically acceptable salt thereof or preventing the occurrence of respiratory depression, apnea, or opioid overdose in a patient who is at a higher-risk of experiencing respiratory depression, apnea, or opioid overdose. Such high-risk patients include patients who are opioid addicts, drug-tolerant patients, or patients who are being treated for opioid use disorder.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In aspects, a patient is human. In aspects, the patient is a patient taking an opioid. In aspect, the patient is a drug-tolerant patient.

“Patient taking an opioid” or “opioid patient” or variants thereof refer to a patient who is taking an opioid and has measurable blood plasma concentration levels of the opioid. In aspects, the patient is taking an opioid by an injectable route. In aspects, the patient is taking an opioid by a non-injectable route, such as pulmonary route (e.g., inhalation, smoking), oral route (e.g., swallowing), nasal route (e.g., snorting), transdermal (e.g., transdermal patch), or rectal route (e.g., suppository). In aspects, the patient taking an opioid is taking the opioid to treat pain.

“Drug-tolerant patient” refers to a patient or subject that is using opioids, including patients that are misusing or abusing opioids and patients that are administered opioids by a health care professional.

A “therapeutically effective amount” is an amount of buprenorphine or a pharmaceutically acceptable salt thereof to accomplish a stated purpose relative to the absence of the compound (e.g. achieve the effect for which it is administered, treat a disease, or reduce one or more symptoms of a disease or condition). An example of a “therapeutically effective amount” is an amount sufficient to contribute to the treatment of a disease (e.g., respiratory depression, apnea, opioid overdose). In aspects, a “therapeutically effective amount” is an amount of buprenorphine that provides or achieves sustained mu-opioid receptor occupancy, thereby inhibiting or preventing mu-opioid receptor occupancy by an opioid, thereby reducing the probability the patient will experience respiratory depression, apnea, or opioid overdose. “Treating” encompasses decreasing the severity of the respiratory depression or apnea or eliminating the respiratory depression or apnea. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Dosages may be varied depending upon the requirements of the patient. The dose administered to a patient, in the context of the present disclosure, should be sufficient to effect a beneficial therapeutic response in the patient. The size of the dose also will be determined by the existence, nature, and extent of any adverse side-effects. Determination of the proper dosage for a particular situation is within the skill of the practitioner. In aspects, a therapeutically effective amount of buprenorphine is the dosage available from commercially-available buprenorphine products, such as SUBLOCADE® (Indivior), SUBUTEX® (Indivior), TEMGESIC® (Indivior), SUBOXONE® tablets (Indivior), SUBOXONE® film (Indivior), BUPRENEX® (Indivior), BELBUCA® (BioDelivery Sciences International, Inc.), BUTRANS® (Purdue Pharma), PROBUPHINE® (Titan Pharmaceuticals), BUVIDAL® (Camurus AB), BRIXADI™ (Braeburn Pharmaceuticals Inc.), and generic equivalents of any of the foregoing. In aspects, a therapeutically effective amount of buprenorphine is the dosage available from commercially-available buprenorphine and naloxone products, such as SUBOXONE® tablets (Indivior), SUBOXONE® film (Indivior), BUNAVAIL® (BioDelivery Sciences International, Inc.), ZUBSOLV® (Orexo), and generic equivalents of any of the foregoing.

The term “administering” means oral administration, administration as a suppository, topical contact, intravenous, subcutaneous, parenteral, intraperitoneal, intramuscular, intrathecal, intranasal or subcutaneous administration, or the implantation of a slow-release device, e.g., a mini-osmotic pump, to a subject. Administration is by any route, including parenteral, transmucosal (e.g., buccal, sublingual, palatal, gingival, nasal, vaginal, rectal), or transdermal. Parenteral administration includes, e.g., intravenous, intramuscular, intra-arteriole, intradermal, subcutaneous, intraperitoneal, intraventricular, and intracranial. In aspects, administering is subcutaneous injection. In aspects, administering is intramuscular injection. In aspects, administering is intravenous. In aspects, administering is intravenous infusion. In aspects, administering is intravenous bolus. In aspects, administering is via sublingual or buccal film.

A dosing regimen that is “once per month” means that the pharmaceutical composition comprising buprenorphine is administered one time in one month and one time in the following month, where each administration of the pharmaceutical composition is separated by one month (e.g., 28-31 days). For example, the pharmaceutical composition comprising buprenorphine may be administered on January 1, and is then administered on February 1 (e.g., +/−3 days); and is then administered on March 1 (e.g., +/−3 days), and so on. As another example, “once per week” means that pharmaceutical composition comprising buprenorphine is administered on a Monday, then is administered 7 days (e.g., +/−1 day) later on the following Monday, and is then administered 7 days (e.g., +/−1 day) later on the following Monday, and so on.

In aspects, the buprenorphine or the pharmaceutically acceptable salt thereof is administered to a patient in any manner known in the art. In aspects, the buprenorphine is administered by intravenous injection. An intravenous injection is also referred to as an intravenous bolus injection. In aspects, the buprenorphine is administered by intravenous infusion. In aspects, the buprenorphine is administered by subcutaneous injection. In aspects, the buprenorphine is administered by intramuscular injection. Such intravenous buprenorphine formulations are commercially available as TEMGESIC® (Indivior), BUPRENEX® (Indivior), and generic equivalents thereof. In aspects, the buprenorphine is administered by subcutaneous injection. Such subcutaneous buprenorphine formulations are commercially available as SUBLOCADE® (Indivior), BUVIDAL® (Camurus AB), BRIXADI™ (Braeburn Pharmaceuticals Inc.), and generic equivalents thereof. In aspects, the buprenorphine is administered through the oral mucosa (e.g., sublingual, buccal). Such oral mucosa formulations include SUBUTEX® (Indivior), SUBOXONE® tablets (Indivior), SUBOXONE® film (Indivior), BELBUCA® (BioDelivery Sciences International, Inc.), BUNAVAIL® (BioDelivery Sciences International, Inc.), ZUBSOLV® (Orexo), and generic equivalents thereof. In aspects, the buprenorphine is administered transdermally. Such transdermal buprenorphine formulations are commercially available as BUTRANS® (Purdue Pharma), and generic equivalents thereof. In aspects, the buprenorphine is administered by an implantable device. Such implantable devices are commercially available as PROBUPHINE® (Titan Pharmaceuticals, Inc.), and generic equivalents thereof.

The term “long-acting injectable pharmaceutical composition” refers to any pharmaceutical composition comprising buprenorphine or a pharmaceutically acceptable salt thereof that provides therapeutic buprenorphine plasma concentration levels (e.g., ≥2 ng/mL) and/or mu-opioid receptor occupancy levels (e.g., at least 70%) in a patient for a sustained period of time. In aspects, a long-acting injectable pharmaceutical composition that is administered once per week, twice per month, once per month, once every two months, once every three months, once every four months, once every five months, once every six months, or once per year. In aspects, the pharmaceutical composition is an injectable pharmaceutical composition that is administered once per week. In aspects, the pharmaceutical composition is an injectable pharmaceutical composition that is administered twice per month (i.e., every other week). In aspects, the pharmaceutical composition is an injectable pharmaceutical composition that is administered once per month. In aspects, the pharmaceutical composition is an injectable pharmaceutical composition that is administered once every two months (i.e., every other month). In aspects, “long-acting injectable pharmaceutical composition” is Formulation A, Formulation B, Formulation C, Formulation D, Formulation E, Formulation F, and variations thereof. In aspects, the long-acting injectable pharmaceutical composition is Formulation A. In aspects, the long-acting injectable pharmaceutical composition is Formulation B. In aspects, the long-acting injectable pharmaceutical composition is Formulation C. In aspects, the long-acting injectable pharmaceutical composition is Formulation D. In aspects, the long-acting injectable pharmaceutical composition is Formulation E. In aspects, the long-acting injectable pharmaceutical composition is Formulation F. Commercially available long-acting injectable pharmaceutical compositions include SUBLOCADE® (Indivior), BUVIDAL® (Camurus AB), BRIXADI™ (Braeburn Pharmaceuticals Inc.), and generic equivalents of each of the foregoing.

The term “sustained” with reference to sustained μ-opioid receptor occupancy or sustained buprenorphine plasma concentration means that the μ-opioid receptor occupancy and/or the buprenorphine plasma concentration remain at a clinically effective level for an extended period of time. The term “extended period of time” means for at least one week. In aspects, the term “extended period of time” means for at least one month. In aspects, the clinically effective level for μ-opioid receptor occupancy is at least 70%. In aspects, the clinically effective level for buprenorphine plasma concentration is 2 ng/mL or more. As an example, a sustained buprenorphine plasma concentration means that the patient has a buprenorphine plasma concentration ≥2 ng/mL for at least one month.

In aspects, “buprenorphine or a pharmaceutically acceptable salt thereof” refers to Formulation A. “Formulation A” is a flowable composition that comprises, consists essentially of, or consists of about 18 wt % buprenorphine free base; about 32 wt % of a 50:50 poly(DL-lactide-co-glycolide) copolymer; and about 50 wt % of N-methyl-2-pyrrolidone. In aspects, the poly(DL-lactide-co-glycolide) copolymer has a carboxy terminal group. In aspects, the poly(DL-lactide-co-glycolide) copolymer has an average molecular weight of about 9,000 Daltons to about 19,000 Daltons. In aspects, the poly(DL-lactide-co-glycolide) copolymer has a carboxy terminal group and an average molecular weight of about 9,000 Daltons to about 19,000 Daltons. Formulation A is also known as SUBLOCADE® (buprenorphine extended-release by Indivior), which is described in U.S. Pat. Nos. 8,921,387, 8,975,270, 9,272,044, 9,498,432, 9,782,402, 9,827,241, and 10,198,218, the disclosures of which are incorporated by reference.

In aspects, “buprenorphine or a pharmaceutically acceptable salt thereof” refers to Formulation B. “Formulation B” is a flowable composition that comprises, consists essentially of, or consists of about 14 wt % to about 22 wt % buprenorphine free base; about 22 wt % to about 42 wt % of a poly(DL-lactide-co-glycolide) copolymer; and about 40 wt % to about 60 wt % of N-methyl-2-pyrrolidone. In aspects, the poly(DL-lactide-co-glycolide) copolymer is 50:50 to 95:5 poly(DL-lactide-co-glycolide) copolymer. In aspects, the poly(DL-lactide-co-glycolide) copolymer is 50:50 to 80:20 poly(DL-lactide-co-glycolide) copolymer. In aspects, the poly(DL-lactide-co-glycolide) copolymer has an average molecular weight of about 5,000 Daltons to about 30,000 Daltons. In aspects, the poly(DL-lactide-co-glycolide) copolymer has a carboxy terminal group. In aspects, the poly(DL-lactide-co-glycolide) copolymer has a carboxy terminal group and an average molecular weight of about 5,000 Daltons to about 30,000 Daltons.

In aspects, “buprenorphine or a pharmaceutically acceptable salt thereof” refers to Formulation C. “Formulation C” is a flowable composition that comprises, consists essentially of, or consists of about 10 wt % to about 30 wt % buprenorphine in the form of the free base or a pharmaceutically acceptable salt; about 10 wt % to about 60 wt % of a poly(DL-lactide-co-glycolide) copolymer; and about 30 wt % to about 70 wt % of N-methyl-2-pyrrolidone. In aspects of Formulation B, the buprenorphine is in the form of the free base. In aspects, the poly(DL-lactide-co-glycolide) copolymer is 50:50 to 95:5 poly(DL-lactide-co-glycolide) copolymer. In aspects, the poly(DL-lactide-co-glycolide) copolymer is 50:50 to 80:20 poly(DL-lactide-co-glycolide) copolymer. In aspects, the poly(DL-lactide-co-glycolide) copolymer has an average molecular weight of about 5,000 Daltons to about 40,000 Daltons. In aspects, the poly(DL-lactide-co-glycolide) copolymer has a carboxy terminal group. In aspects, the poly(DL-lactide-co-glycolide) copolymer has a carboxy terminal group and an average molecular weight of about 5,000 Daltons to about 40,000 Daltons.

In aspects, “buprenorphine or a pharmaceutically acceptable salt thereof” refers to Formulation D. “Formulation D” is a flowable composition that comprises, consists essentially of, or consists of: (i) at least one biodegradable polymer; (ii) at least one organic solvent which comprises an amide, an ester, a carbonate, a ketone, a lactam, an ether, a sulfonyl, or a combination thereof; and (iii) about 5 wt % to about 30 wt % of buprenorphine in the form of a free base or pharmaceutically acceptable salt. In aspects, the buprenorphine is in the form of a free base. In other aspects, the buprenorphine is present in an amount from about 10 wt % to about 25 wt %; or in an amount from about 15 wt % to about 20 wt %. In aspects, the organic solvent is present in an amount of about 30 wt % to about 70 wt %; or in an amount of about 40 wt % to about 60 wt %. In aspects, the organic solvent is N-methyl-2-pyrrolidone, 2-pyrrolidone, propylene glycol, ethanol, acetone, tetrahydrofurfuryl alcohol, dimethyl isosorbide, acetic acid, lactic acid, methyl lactate, ethyl lactate, monomethyl succinate acid, monomethyl citric acid, glycofurol, glycerol formal, isopropylidene glycol, 2,2-dimethyl-1,3-dioxolone-4-methanol, dimethylformamide, dimethylacetamide, N,N-dimethylformamide, propylene carbonate, triacetin, dimethylsulfoxide, dimethyl sulfone, epsilon-caprolactone, butyrolactone, caprolactam, and a mixture of two or more thereof. In other aspects, the organic solvent is N-methyl-2-pyrrolidone, dimethyl sulfoxide, N,N-dimethylformamide, acetone, ethyl acetate, tributyl citrate, diethyl succinate, triethyl citrate, acetyl tributyl citrate, glyceryl triacetate, dimethylacetamide, epsilon-caprolactone, or a combination of two or more thereof. In aspects, the organic solvent is N-methyl-2-pyrrolidone. In aspects, the organic solvent is dimethyl sulfoxide. The term “biodegradable polymer” refers to any polymer that can degrade in vivo and be eliminated from a patient's body. In other aspects, the biodegradable polymer is present in an amount of about 10 wt % to about 90 wt %; or in an amount of about 10 wt % to about 80 wt %; or in an amount of about 10 wt % to about 70 wt %; or in an amount of about 10 wt % to about 60 wt %; or in an amount of about 10 wt % to about 50 wt %; or in an amount of about 20 wt % to about 40 wt %. In aspects, the polymer is a polylactide, a polyglycolide, a polycaprolactone, a copolymer thereof, a terpolymer thereof, any combination thereof, or a mixture of two or more thereof. In aspects, the polymer is a poly(DL-lactide-co-glycolide) copolymer. The polymer, such as the poly(DL-lactide-co-glycolide) copolymer, can have an average molecular weight of about 1,000 Daltons to about 50,000 Daltons; or from about 5,000 Daltons to about 40,000 Daltons; or from about 5,000 Daltons to about 30,000 Daltons; or from about 5,000 Daltons to about 20,000 Daltons; or from about 10,000 Daltons to about 20,000 Daltons. The poly(DL-lactide-co-glycolide) copolymer can be a 50:50 to 95:5 poly(DL-lactide-co-glycolide) copolymer; or a 50:50 to 80:20 poly(DL-lactide-co-glycolide) copolymer; or a 50:50 poly(DL-lactide-co-glycolide) copolymer. In aspects, the poly(DL-lactide-co-glycolide) copolymer has a carboxy terminal group.

In aspects, the buprenorphine formulations comprise from about 295 mg to about 305 mg of buprenorphine, or about 300 mg of buprenorphine. In aspects, the buprenorphine formulations comprise from about 95 mg to about 105 mg buprenorphine, or about 100 mg buprenorphine.

In aspects, “buprenorphine or a pharmaceutically acceptable salt thereof” refers to Formulation E. “Formulation E” is a flowable composition that comprises, consists essentially of, or consists of buprenorphine and one or more pharmaceutically acceptable excipients. In aspects, Formulation E comprises a pharmaceutically acceptable salt of buprenorphine, a monosaccharide, and water. In aspects, the monosaccharide is glucose, dextrose, fructose, levulose, galactose, ribose, or xylose. In aspects, Formulation E comprises a pharmaceutically acceptable salt of buprenorphine, dextrose, hydrochloric acid, and water. In aspects, Formulation E is known as TEMGESIC® (Indivior).

In aspects, “buprenorphine or a pharmaceutically acceptable salt thereof” refers to Formulation F. “Formulation F” is a flowable composition that comprises, consists essentially of, or consists of buprenorphine, a phospholipid, a neutral diacyl lipid, and an organic solvent. In aspects, the organic solvent is N-methyl-2-pyrrolidone, 2-pyrrolidone, propylene glycol, ethanol, acetone, tetrahydrofurfuryl alcohol, dimethyl isosorbide, acetic acid, lactic acid, methyl lactate, ethyl lactate, monomethyl succinate acid, monomethyl citric acid, glycofurol, glycerol formal, isopropylidene glycol, 2,2-dimethyl-1,3-dioxolone-4-methanol, dimethylformamide, dimethylacetamide, N,N-dimethylformamide, propylene carbonate, triacetin, dimethylsulfoxide, dimethyl sulfone, epsilon-caprolactone, butyrolactone, caprolactam, and a mixture of two or more thereof. In other aspects, the organic solvent is N-methyl-2-pyrrolidone, dimethyl sulfoxide, N,N-dimethylformamide, acetone, ethyl acetate, tributyl citrate, diethyl succinate, triethyl citrate, acetyl tributyl citrate, glyceryl triacetate, dimethylacetamide, epsilon-caprolactone, or a combination of two or more thereof. In aspects, the phospholipid is phosphatidylcholine. In aspects, the phosphatidylcholine is soybean phosphatidylcholine. In aspects, the neutral diacyl lipid is glycerol dioleate. In aspects, the organic solvent is ethanol. In aspects, the organic solvent is N-methyl-2-pyrrolidone (NMP). In aspects, the organic solvent is isopropanol. In aspects, the organic solvent is dimethyl sulfoxide. In aspects, the organic solvent is ethanol, NMP, isopropanol, or a combination of two or more thereof. In aspects, the organic solvent is ethanol, NMP, 2-pyrrolidone, dimethylacetamide, isopropanol, benzyl alcohol, propylene glycol, benzyl benzoate, dimethyl sulfoxide, or a combination of two or more thereof. In aspects, Formulaion F further comprises tocopherol. In aspects, Formulation F comprises a dose of 8 mg, 16 mg, 24 mg, 32 mg, 64 mg, 96 mg, or 128 mg of buprenorphine. In aspects, the ethanol is anhydrous ethanol. In aspects, Formulation F comprises from about 4 wt % to about 10 wt % buprenorphine, about 30 wt % to about 50 wt % of phosphatidylcholine, about 30 wt % to about 50 wt % of glycerol dioleate, and about 5 wt % to about 15 wt % of ethanol, NMP, or a combination thereof. In aspects, Formulation F comprises from about 4 wt % to about 8 wt % buprenorphine, about 40 wt % to about 45 wt % of phosphatidylcholine, about 40 wt % to about 45 wt % of glycerol dioleate, and about 8 wt % to about 12 wt % of ethanol, NMP, or a combination thereof. In aspects, Formulation F is known as BUVIDAL® (Camurus AB, Sweden) or BRIXADI™ (Braeburn Inc.). Formulation F is described, e.g., in U.S. Pat. Nos. 8,236,755 and 9,937,164, the disclosures of which are incorporated by reference herein.

The phrase “average molecular weight” refers to the weight average molecular weight of a polymer as determined by gel permeation chromatography (also known as GPC or size exclusion chromatography (SEC)) using tetrahydrofuran (THF) as the solvent and using a molecular weight calibration curve using polystyrene standards.

The term “pharmaceutically acceptable salts” is meant to include salts of the active compounds that are prepared with relatively nontoxic acids or bases, depending on the particular substituents found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

As described herein, opioids can induce respiratory depression, apnea, or overdose in a patient. The patient may be misusing or abusing the opioids or the patient may be prescribed the opioids. Either way, it would be beneficial if patients taking opioids could: (1) reduce the probability or incidence of experiencing respiratory depression or apnea caused by the opioids, (2) reduce the extent or degree of the respiratory depression or apnea that is caused by the opioids, or (3) prevent the occurrence of respiratory depression or apnea that could be caused by opioids. Such results can be achieved by administering buprenorphine or a pharmaceutically acceptable salt thereof to the patient prior to the occurrence of the respiratory depression, apnea, or overdose. Similarly, patients known to be abusing opioids (e.g., patients diagnosed with or being treated for opioid use disorder) should concurrently be treated with buprenorphine or a pharmaceutically acceptable salt thereof, because buprenorphine will (1) reduce the probability or incidence of experiencing respiratory depression or apnea caused by the opioids, (2) reduce the extent or degree of the respiratory depression or apnea that is caused by the opioids, or (3) prevent the occurrence of respiratory depression or apnea that could be caused by opioids.

The disclosure provides methods of treating or preventing opioid-induced respiratory depression in a patient in need thereof comprising administering to the patient a therapeutically effective amount of buprenorphine or a pharmaceutically acceptable salt thereof. The disclosure provides methods of reducing the incidence of opioid-induced respiratory depression in a patient in need thereof comprising administering to the patient a therapeutically effective amount of buprenorphine or a pharmaceutically acceptable salt thereof. In aspects, the buprenorphine or the pharmaceutically acceptable salt thereof is administered prior to occurrence of the opioid-induced respiratory depression. In aspects, the method is for treating opioid-induced respiratory depression in a patient. In aspects, the method is for preventing opioid-induced respiratory depression in a patient. In aspects, the buprenorphine or pharmaceutically acceptable salt thereof is a long-acting injectable pharmaceutical composition comprising buprenorphine or pharmaceutically acceptable salt thereof. In aspects, the long-acting injectable pharmaceutical composition is Formulation A, Formulation B, Formulation C, Formulation D, Formulation E, or Formulation F. In aspects, the method comprises administering Formulation A. In aspects, the method comprises administering Formulation B. In aspects, the method comprises administering Formulation C. In aspects, the method comprises administering Formulation D. In aspects, the method comprises administering Formulation E. In aspects, the method comprises administering Formulation F. In aspects, the patient is taking the opioid prior to administration of the buprenorphine. In aspects, the patient is taking the opioid for any reason. In aspects, the patient is taking the opioid for pain. In aspects, the opioid is fentanyl. In aspects, the opioid is a fentanyl analogue. In aspects, the opioid is carfentanil. In aspects, the opioid is a carfentanil analogue. In aspects, the opioid is heroin. In aspects, the opioid is fentanyl, carfentanil, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or a combination of two or more thereof. In aspects, the buprenorphine or the pharmaceutically acceptable salt thereof is administered parenterally. In aspects, the buprenorphine or the pharmaceutically acceptable salt thereof is administered by intravenous injection. In aspects, the buprenorphine or the pharmaceutically acceptable salt thereof is administered by intravenous infusion. In aspects, the buprenorphine or the pharmaceutically acceptable salt thereof is administered subcutaneously. In aspects, the buprenorphine or the pharmaceutically acceptable salt thereof is administered transmucosally. In aspects, the buprenorphine or the pharmaceutically acceptable salt thereof is administered sublingually. In aspects, the buprenorphine or the pharmaceutically acceptable salt thereof is administered bucally. In aspects, the buprenorphine or the pharmaceutically acceptable salt thereof is administered transdermally. In aspects, the buprenorphine or the pharmaceutically acceptable salt thereof is administered via an implanted device. In aspects, the therapeutically effective amount of buprenorphine or the pharmaceutically acceptable salt thereof achieves sustained μ-opioid receptor occupancy of at least 70%. In aspects, the therapeutically effective amount of buprenorphine is an amount sufficient to provide a buprenorphine plasma concentration of about 2 ng/mL or more. In aspects, the therapeutically effective amount of buprenorphine is an amount sufficient to provide a buprenorphine plasma concentration of about 5 ng/mL or more. In aspects, the therapeutically effective amount of buprenorphine is an amount sufficient to provide a buprenorphine plasma concentration from about 1 ng/mL to about 20 ng/mL. In aspects, the therapeutically effective amount of buprenorphine is an amount sufficient to provide a buprenorphine plasma concentration from about 2 ng/mL to about 20 ng/mL. In aspects, the therapeutically effective amount of buprenorphine is an amount sufficient to provide a buprenorphine plasma concentration from about 2 ng/mL to about 15 ng/mL. In aspects, the therapeutically effective amount of buprenorphine is an amount sufficient to provide a buprenorphine plasma concentration from about 2 ng/mL to about 12 ng/mL. In aspects, the therapeutically effective amount of buprenorphine is an amount sufficient to provide a buprenorphine plasma concentration from about 2 ng/mL to about 10 ng/mL. In aspects, the therapeutically effective amount of buprenorphine is an amount sufficient to provide a buprenorphine plasma concentration from about 2 ng/mL to about 9 ng/mL. In aspects, the therapeutically effective amount of buprenorphine is an amount sufficient to provide a buprenorphine plasma concentration from about 2 ng/mL to about 8 ng/mL. In aspects, the therapeutically effective amount of buprenorphine is an amount sufficient to provide a buprenorphine plasma concentration from about 2 ng/mL to about 7 ng/mL. In aspects, the therapeutically effective amount of buprenorphine is an amount sufficient to provide a buprenorphine plasma concentration from about 2 ng/mL to about 6 ng/mL. In aspects, the therapeutically effective amount of buprenorphine is an amount sufficient to provide a buprenorphine plasma concentration from about 2 ng/mL to about 5 ng/mL. In aspects, the therapeutically effective amount of buprenorphine is an amount sufficient to provide a buprenorphine plasma concentration of about 2 ng/mL. In aspects, the therapeutically effective amount of buprenorphine is an amount sufficient to provide a buprenorphine plasma concentration of about 5 ng/mL. In aspects, the patient is a drug-tolerant patient. In aspects, the patient is being treated for opioid use disorder. In aspects, the patient is concurrently experiencing opioid-induced respiratory depression and an opioid overdose. In aspects, the opioid-induced respiratory depression precedes an opioid overdose. In aspects, the opioid-induced respiratory depression precedes opioid-induced apnea. In aspects, the opioid-induced respiratory depression precedes opioid-induced apnea and an opioid overdose.

The disclosure provides methods of treating or preventing fentanyl-induced respiratory depression or reducing the incidence of fentanyl-induced respiratory depression in a patient in need thereof comprising subcutaneously administering to the patient a therapeutically effective amount of a long-acting injectable pharmaceutical composition comprising buprenorphine or pharmaceutically acceptable salt thereof. In aspects, the long-acting injectable pharmaceutical composition is Formulation A, Formulation B, Formulation C, Formulation D, Formulation E, or Formulation F. In aspects, the method comprises administering Formulation A. In aspects, the method comprises administering Formulation B. In aspects, the method comprises administering Formulation C. In aspects, the method comprises administering Formulation D. In aspects, the method comprises administering Formulation E. In aspects, the method comprises administering Formulation F. In aspects, the buprenorphine is administered to the patient prior to occurrence of the fentanyl-induced respiratory depression. In aspects, the method is for treating fentanyl-induced respiratory depression in a patient. In aspects, the method is for preventing fentanyl-induced respiratory depression in a patient. In aspects, the method is for reducing the incidence of fentanyl-induced respiratory depression. In aspects, the patient is taking fentanyl prior to administration of the buprenorphine. In aspects, the patient is taking fentanyl for pain. In aspects, the therapeutically effective amount of buprenorphine achieves sustained μ-opioid receptor occupancy of at least 70%. In aspects, the therapeutically effective amount of buprenorphine is an amount sufficient to provide a buprenorphine plasma concentration of about 2 ng/mL or more. In aspects, the therapeutically effective amount of buprenorphine is an amount sufficient to provide a buprenorphine plasma concentration of about 5 ng/mL or more. In aspects, the therapeutically effective amount of buprenorphine is an amount sufficient to provide a buprenorphine plasma concentration from about 1 ng/mL to about 20 ng/mL. In aspects, the therapeutically effective amount of buprenorphine is an amount sufficient to provide a buprenorphine plasma concentration from about 2 ng/mL to about 20 ng/mL. In aspects, the therapeutically effective amount of buprenorphine is an amount sufficient to provide a buprenorphine plasma concentration from about 2 ng/mL to about 15 ng/mL. In aspects, the therapeutically effective amount of buprenorphine is an amount sufficient to provide a buprenorphine plasma concentration from about 2 ng/mL to about 12 ng/mL. In aspects, the therapeutically effective amount of buprenorphine is an amount sufficient to provide a buprenorphine plasma concentration from about 2 ng/mL to about 10 ng/mL. In aspects, the therapeutically effective amount of buprenorphine is an amount sufficient to provide a buprenorphine plasma concentration from about 2 ng/mL to about 9 ng/mL. In aspects, the therapeutically effective amount of buprenorphine is an amount sufficient to provide a buprenorphine plasma concentration from about 2 ng/mL to about 8 ng/mL. In aspects, the therapeutically effective amount of buprenorphine is an amount sufficient to provide a buprenorphine plasma concentration from about 2 ng/mL to about 7 ng/mL. In aspects, the therapeutically effective amount of buprenorphine is an amount sufficient to provide a buprenorphine plasma concentration from about 2 ng/mL to about 6 ng/mL. In aspects, the therapeutically effective amount of buprenorphine is an amount sufficient to provide a buprenorphine plasma concentration from about 2 ng/mL to about 5 ng/mL. In aspects, the therapeutically effective amount of buprenorphine is an amount sufficient to provide a buprenorphine plasma concentration of about 2 ng/mL. In aspects, the therapeutically effective amount of buprenorphine is an amount sufficient to provide a buprenorphine plasma concentration of about 5 ng/mL. In aspects, the therapeutically effective amount of buprenorphine is an amount sufficient to reduce, treat, or prevent fentanyl-induced respiratory depression, but the therapeutically effective amount of buprenorphine is not an amount sufficient to provide analgesia (pain relief). In aspects, the patient is a drug-tolerant patient. In aspects, the patient is being treated for opioid use disorder. In aspects, the patient is concurrently experiencing fentanyl-induced respiratory depression and a fentanyl overdose. In aspects, the fentanyl-induced respiratory depression precedes a fentanyl overdose. In aspects, the fentanyl-induced respiratory depression precedes fentanyl-induced apnea. In aspects, the fentanyl-induced respiratory depression precedes fentanyl-induced apnea and a fentanyl overdose.

The disclosure provides methods of treating or preventing opioid-induced apnea or reducing the incidence of opioid-induced apnea in a patient in need thereof comprising administering to the patient a therapeutically effective amount of buprenorphine or a pharmaceutically acceptable salt thereof. In aspects, the buprenorphine or the pharmaceutically acceptable salt thereof is administered prior to occurrence of the opioid-induced apnea. In aspects, the method is for treating opioid-induced apnea in a patient. In aspects, the method is for preventing opioid-induced apnea in a patient. In aspects, the method is for reducing the incidence of opioid-induced apnea. In aspects, the buprenorphine or pharmaceutically acceptable salt thereof is a long-acting injectable pharmaceutical composition comprising buprenorphine or pharmaceutically acceptable salt thereof. In aspects, the long-acting injectable pharmaceutical composition is Formulation A, Formulation B, Formulation C, Formulation D, Formulation E, or Formulation F. In aspects, the method comprises administering Formulation A. In aspects, the method comprises administering Formulation B. In aspects, the method comprises administering Formulation C. In aspects, the method comprises administering Formulation D. In aspects, the method comprises administering Formulation E. In aspects, the method comprises administering Formulation F. In aspects, the patient is taking the opioid prior to administration of the buprenorphine. In aspects, the patient is taking the opioid for pain. In aspects, the opioid is fentanyl. In aspects, the opioid is a fentanyl analogue. In aspects, the opioid is carfentanil. In aspects, the opioid is a carfentanil analogue. In aspects, the opioid is heroin. In aspects, the opioid is fentanyl, carfentanil, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or a combination of two or more thereof. In aspects, the buprenorphine or the pharmaceutically acceptable salt thereof is administered parenterally. In aspects, the buprenorphine or the pharmaceutically acceptable salt thereof is administered by intravenous injection. In aspects, the buprenorphine or the pharmaceutically acceptable salt thereof is administered by intravenous infusion. In aspects, the buprenorphine or the pharmaceutically acceptable salt thereof is administered subcutaneously. In aspects, the buprenorphine or the pharmaceutically acceptable salt thereof is administered transmucosally. In aspects, the buprenorphine or the pharmaceutically acceptable salt thereof is administered sublingually. In aspects, the buprenorphine or the pharmaceutically acceptable salt thereof is administered bucally. In aspects, the buprenorphine or the pharmaceutically acceptable salt thereof is administered transdermally. In aspects, the buprenorphine or the pharmaceutically acceptable salt thereof is administered via an implanted device. In aspects, the therapeutically effective amount of buprenorphine or the pharmaceutically acceptable salt thereof achieves sustained μ-opioid receptor occupancy of at least 70%. In aspects, the therapeutically effective amount of buprenorphine is an amount sufficient to provide a buprenorphine plasma concentration of about 2 ng/mL or more. In aspects, the therapeutically effective amount of buprenorphine is an amount sufficient to provide a buprenorphine plasma concentration of about 5 ng/mL or more. In aspects, the therapeutically effective amount of buprenorphine is an amount sufficient to provide a buprenorphine plasma concentration from about 1 ng/mL to about 20 ng/mL. In aspects, the therapeutically effective amount of buprenorphine is an amount sufficient to provide a buprenorphine plasma concentration from about 2 ng/mL to about 20 ng/mL. In aspects, the therapeutically effective amount of buprenorphine is an amount sufficient to provide a buprenorphine plasma concentration from about 2 ng/mL to about 15 ng/mL. In aspects, the therapeutically effective amount of buprenorphine is an amount sufficient to provide a buprenorphine plasma concentration from about 2 ng/mL to about 12 ng/mL. In aspects, the therapeutically effective amount of buprenorphine is an amount sufficient to provide a buprenorphine plasma concentration from about 2 ng/mL to about 10 ng/mL. In aspects, the therapeutically effective amount of buprenorphine is an amount sufficient to provide a buprenorphine plasma concentration from about 2 ng/mL to about 9 ng/mL. In aspects, the therapeutically effective amount of buprenorphine is an amount sufficient to provide a buprenorphine plasma concentration from about 2 ng/mL to about 8 ng/mL. In aspects, the therapeutically effective amount of buprenorphine is an amount sufficient to provide a buprenorphine plasma concentration from about 2 ng/mL to about 7 ng/mL. In aspects, the therapeutically effective amount of buprenorphine is an amount sufficient to provide a buprenorphine plasma concentration from about 2 ng/mL to about 6 ng/mL. In aspects, the therapeutically effective amount of buprenorphine is an amount sufficient to provide a buprenorphine plasma concentration from about 2 ng/mL to about 5 ng/mL. In aspects, the therapeutically effective amount of buprenorphine is an amount sufficient to provide a buprenorphine plasma concentration of about 2 ng/mL. In aspects, the therapeutically effective amount of buprenorphine is an amount sufficient to provide a buprenorphine plasma concentration of about 5 ng/mL. In aspects, the patient is a drug-tolerant patient. In aspects, the patient is being treated for opioid use disorder. In aspects, the therapeutically effective amount of buprenorphine is an amount sufficient to reduce, treat, or prevent opioid-induced apnea, but the therapeutically effective amount of buprenorphine is not an amount sufficient to provide analgesia (pain relief). In aspects, the patient is concurrently experiencing opioid-induced apnea and an opioid overdose. In aspects, the opioid-induced apnea precedes an opioid overdose. In aspects, the opioid-induced apnea precedes opioid-induced apnea. In aspects, the opioid-induced apnea precedes opioid-induced apnea and an opioid overdose.

Unlike drugs like naloxone, which can immediately reverse the effects of an opioid overdose, the administration of buprenorphine prior to an opioid overdose can lessen the effects of an opioid overdose or reduce the probability of an opioid overdose occurring because the buprenorphine reduces respiratory depression and apnea associated with opioids. Thus, the disclosure provides methods of treating or preventing an opioid overdose or reducing the incidence of an opioid overdose in a patient in need thereof comprising administering to the patient a therapeutically effective amount of buprenorphine or a pharmaceutically acceptable salt thereof. In aspects, the patient is taking the opioid prior to administration of the buprenorphine. In aspects, the buprenorphine or pharmaceutically acceptable salt thereof is a long-acting injectable pharmaceutical composition comprising buprenorphine or pharmaceutically acceptable salt thereof. In aspects, the long-acting injectable pharmaceutical composition is Formulation A, Formulation B, Formulation C, Formulation D, Formulation E, or Formulation F. In aspects, the method comprises administering Formulation A. In aspects, the method comprises administering Formulation B. In aspects, the method comprises administering Formulation C. In aspects, the method comprises administering Formulation D. In aspects, the method comprises administering Formulation E. In aspects, the method comprises administering Formulation F. In aspects, the buprenorphine or the pharmaceutically acceptable salt thereof is administered prior to occurrence of the opioid overdose. In aspects, the method is for treating an opioid overdose in a patient. In aspects, the method is for preventing an opioid overdose in a patient. In aspects, the method is for reducing the incidence of an opioid overdose. In aspects, the patient is taking the opioid for pain. By reducing the incidence or severity of respiratory depression or apnea, the buprenorphine reduces the impact of an opioid overdose or prevents an opioid overdose from occurring. In aspects, the method of treating the opioid overdose is a method of reducing the impact of an opioid overdose. In aspects, the method of treating the opioid overdose is a method of reducing opioid-induced respiratory depression associated with the opioid overdose. In aspects, the method of treating the opioid overdose is a method of reducing opioid-induced apnea associated with the opioid overdose. In aspects, the method of treating the opioid overdose is a method of reducing opioid-induced respiratory depression and opioid-induced apnea associated with the opioid overdose. In aspects, the opioid is fentanyl. In aspects, the opioid is a fentanyl analogue. In aspects, the opioid is carfentanil. In aspects, the opioid is a carfentanil analogue. In aspects, the opioid is heroin. In aspects, the opioid is fentanyl, carfentanil, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or a combination of two or more thereof. In aspects, the buprenorphine or the pharmaceutically acceptable salt thereof is administered intravenously. In aspects, the buprenorphine or the pharmaceutically acceptable salt thereof is administered parenterally. In aspects, the buprenorphine or the pharmaceutically acceptable salt thereof is administered by intravenous injection. In aspects, the buprenorphine or the pharmaceutically acceptable salt thereof is administered by intravenous infusion. In aspects, the buprenorphine or the pharmaceutically acceptable salt thereof is administered subcutaneously. In aspects, the buprenorphine or the pharmaceutically acceptable salt thereof is administered transmucosally. In aspects, the buprenorphine or the pharmaceutically acceptable salt thereof is administered sublingually. In aspects, the buprenorphine or the pharmaceutically acceptable salt thereof is administered bucally. In aspects, the buprenorphine or the pharmaceutically acceptable salt thereof is administered transdermally. In aspects, the buprenorphine or the pharmaceutically acceptable salt thereof is administered via an implanted device. In aspects, the therapeutically effective amount of buprenorphine or the pharmaceutically acceptable salt thereof achieves sustained μ-opioid receptor occupancy of at least 70%. In aspects, the therapeutically effective amount of buprenorphine is an amount sufficient to provide a buprenorphine plasma concentration of about 2 ng/mL or more. In aspects, the therapeutically effective amount of buprenorphine is an amount sufficient to provide a buprenorphine plasma concentration of about 5 ng/mL or more. In aspects, the therapeutically effective amount of buprenorphine is an amount sufficient to provide a buprenorphine plasma concentration from about 1 ng/mL to about 20 ng/mL. In aspects, the therapeutically effective amount of buprenorphine is an amount sufficient to provide a buprenorphine plasma concentration from about 2 ng/mL to about 20 ng/mL. In aspects, the therapeutically effective amount of buprenorphine is an amount sufficient to provide a buprenorphine plasma concentration from about 2 ng/mL to about 15 ng/mL. In aspects, the therapeutically effective amount of buprenorphine is an amount sufficient to provide a buprenorphine plasma concentration from about 2 ng/mL to about 12 ng/mL. In aspects, the therapeutically effective amount of buprenorphine is an amount sufficient to provide a buprenorphine plasma concentration from about 2 ng/mL to about 10 ng/mL. In aspects, the therapeutically effective amount of buprenorphine is an amount sufficient to provide a buprenorphine plasma concentration from about 2 ng/mL to about 9 ng/mL. In aspects, the therapeutically effective amount of buprenorphine is an amount sufficient to provide a buprenorphine plasma concentration from about 2 ng/mL to about 8 ng/mL. In aspects, the therapeutically effective amount of buprenorphine is an amount sufficient to provide a buprenorphine plasma concentration from about 2 ng/mL to about 7 ng/mL. In aspects, the therapeutically effective amount of buprenorphine is an amount sufficient to provide a buprenorphine plasma concentration from about 2 ng/mL to about 6 ng/mL. In aspects, the therapeutically effective amount of buprenorphine is an amount sufficient to provide a buprenorphine plasma concentration from about 2 ng/mL to about 5 ng/mL. In aspects, the therapeutically effective amount of buprenorphine is an amount sufficient to provide a buprenorphine plasma concentration of about 2 ng/mL. In aspects, the therapeutically effective amount of buprenorphine is an amount sufficient to provide a buprenorphine plasma concentration of about 5 ng/mL. In aspects, the patient is a drug-tolerant patient. In aspects, the patient is being treated for opioid use disorder. In aspects, the therapeutically effective amount of buprenorphine is an amount sufficient to reduce, treat, or prevent an opioid overdose, but the therapeutically effective amount of buprenorphine is not an amount sufficient to provide analgesia (pain relief).

### Embodiments 1 to 57

Embodiment 1. A method of treating or preventing fentanyl-induced respiratory depression in a patient in need thereof, the method comprising administering to the patient a therapeutically effective amount of a pharmaceutical composition comprising buprenorphine free base, a poly(lactide-co-glycolide) copolymer, and N-methyl-2-pyrrolidone, to treat or prevent fentanyl-induced respiratory depression.

Embodiment 2. The method of Embodiment 1, comprising administering the pharmaceutical composition to the patient once per month by subcutaneous injection.

Embodiment 3. The method of Embodiment 1 or 2, wherein the pharmaceutical composition comprises about 18 wt % buprenorphine free base, about 32 wt % of a 50:50 poly(DL-lactide-co-glycolide) copolymer, and about 50 wt % of N-methyl-2-pyrrolidone.

Embodiment 4. The method of any one of Embodiments 1 to 3, wherein the pharmaceutical composition comprises about 100 mg of buprenorphine free base or about 300 mg of buprenorphine free base.

Embodiment 5. The method of any one of Embodiments 1 to 4, wherein the pharmaceutical composition is administered prior to occurrence of fentanyl-induced respiratory depression.

Embodiment 6. The method of any one of Embodiments 1 to 5, further comprising treating the patient for opioid use disorder.

Embodiment 7. A method of treating or preventing opioid-induced respiratory depression or reducing the incidence of opioid-induced respiratory depression in a patient in need thereof, the method comprising administering to the patient a therapeutically effective amount of a pharmaceutical composition comprising buprenorphine or a pharmaceutically acceptable salt thereof.

Embodiment 8. The method of Embodiment 7 for treating or preventing opioid-induced respiratory depression.

Embodiment 9. The method of Embodiment 7 for reducing the incidence of opioid-induced respiratory depression.

Embodiment 10. The method of any one of Embodiments 7 to 9, wherein the opioid is fentanyl, a fentanyl analogue, carfentanil, a carfentanil analogue, heroin, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or a combination of two or more thereof

Embodiment 11. The method of any one of Embodiments 7 to 9, wherein the opioid is acetylfentanyl, butyrfentanyl, para-tolylfentanyl, 3-methylfentanyl, α-methylfentanyl, mefentanyl, phenaridine, ohmefentanyl, or mirfentanil.

Embodiment 12. The method of any one of Embodiments 7 to 9, wherein the opioid is sufentanil, remifentanil, alfentanil, lofentanil, brifentanil, or trefentanil.

Embodiment 13. The method of any one of Embodiments 7 to 9, wherein the opioid is fentanyl.

Embodiment 14. The method of any one of Embodiments 7 to 9, wherein the opioid is carfentanil.

Embodiment 15. The method of any one of Embodiments 7 to 14, comprising administering the pharmaceutical composition to the patient once per week by subcutaneous injection.

Embodiment 16. The method of any one of Embodiments 7 to 14, comprising administering the pharmaceutical composition to the patient twice per month by subcutaneous injection.

Embodiment 17. The method of any one of Embodiments 7 to 14, comprising administering the pharmaceutical composition to the patient once per month by subcutaneous injection.

Embodiment 18. The method of any one of Embodiments 7 to 14, comprising administering the pharmaceutical composition to the patient once every two months by subcutaneous injection.

Embodiment 19. The method of any one of Embodiments 7 to 14, comprising parenterally administering the pharmaceutical composition to the patient.

Embodiment 20. The method of any one of Embodiments 7 to 19, wherein the pharmaceutical composition is a long-acting pharmaceutical composition.

Embodiment 21. The method of any one of Embodiments 7 to 20, wherein the pharmaceutical composition comprises buprenorphine, a poly(lactide-co-glycolide) copolymer, and N-methyl-2-pyrrolidone.

Embodiment 22. The method of Embodiment 21, wherein the pharmaceutical composition comprises about 18 wt % buprenorphine free base, about 32 wt % of a 50:50 poly(DL-lactide-co-glycolide) copolymer, and about 50 wt % of N-methyl-2-pyrrolidone.

Embodiment 23. The method of any one of Embodiments 7 to 20, wherein the pharmaceutical composition comprises (i) buprenorphine, (ii) phosphatidylcholine, (iii) glycerol dioleate, and (iv) an organic solvent. In aspects, the organic solvent is ethanol, N-methyl-2-pyrrlidone, or a combination thereof.

Embodiment 24. The method of any one of Embodiments 7 to 20, wherein the pharmaceutical composition comprises buprenorphine, dextrose, and water.

Embodiment 25. The method of any one of Embodiments 7 to 24, wherein the pharmaceutical composition is administered to the patient prior to occurrence of opioid-induced respiratory depression.

Embodiment 26. The method of any one of Embodiments 7 25, further comprising treating the patient for opioid use disorder.

Embodiment 27. The method of any one of Embodiments 1 to 26, wherein the therapeutically effective amount of buprenorphine provides a sustained buprenorphine plasma concentration of about 2 ng/mL or more.

Embodiment 28. A method of treating or preventing opioid-induced apnea, reducing the incidence of opioid-induced apnea, treating or preventing an opioid overdose, or reducing the incidence of opioid overdose in a patient in need thereof, the method comprising administering to the patient a therapeutically effective amount of a pharmaceutical composition comprising buprenorphine or a pharmaceutically acceptable salt thereof.

Embodiment 29. The method of Embodiment 28, for treating or preventing opioid-induced apnea.

Embodiment 30. The method of Embodiment 28, for reducing the incidence of opioid-induced apnea.

Embodiment 31. The method of Embodiment 28, for treating or preventing an opioid overdose.

Embodiment 32. The method of Embodiment 28, for reducing the incidence of opioid overdose.

Embodiment 33. The method of any one of Embodiments 28 to 32, wherein the opioid is heroin, butorphanol, oxycodone, hydrocodone, hydromorphone, levorphanol, oxymorphone, opium, meperidine, morphine, codeine, dihydrocodeine, methadone, pentazocine, tapentadol, tramadol, or a combination of two or more thereof

Embodiment 34. The method of any one of Embodiments 28 to 32, wherein the opioid is fentanyl, acetylfentanyl, butyrfentanyl, para-tolylfentanyl, 3-methylfentanyl, α-methylfentanyl, mefentanyl, phenaridine, ohmefentanyl, or mirfentanil.

Embodiment 35. The method of any one of Embodiments 28 to 32, wherein the opioid is fentanyl.

Embodiment 36. The method of any one of Embodiments 28 to 32, wherein the opioid is sufentanil, remifentanil, alfentanil, lofentanil, brifentanil, or trefentanil.

Embodiment 37. The method of any one of Embodiments 28 to 32, wherein the opioid is carfentanil.

Embodiment 38. The method of any one of Embodiments 28 to 37, comprising administering the pharmaceutical composition to the patient once per week by subcutaneous injection.

Embodiment 39. The method of any one of Embodiments 28 to 37, comprising administering the pharmaceutical composition to the patient once per month by subcutaneous injection.

Embodiment 40. The method of any one of Embodiments 28 to 37, comprising administering the pharmaceutical composition to the patient once every two months by subcutaneous injection.

Embodiment 41. The method of any one of Embodiments 28 to 37, comprising parenterally administering the pharmaceutical composition to the patient.

Embodiment 42. The method of any one of Embodiments 28 to 41, wherein the pharmaceutical composition is a long-acting pharmaceutical composition.

Embodiment 43. The method of any one of Embodiments 28 to 42, wherein the pharmaceutical composition comprises buprenorphine, a poly(lactide-co-glycolide) copolymer, and N-methyl-2-pyrrolidone.

Embodiment 44. The method of Embodiment 43, wherein the pharmaceutical composition comprises about 18 wt % buprenorphine free base, about 32 wt % of a 50:50 poly(DL-lactide-co-glycolide) copolymer, and about 50 wt % of N-methyl-2-pyrrolidone.

Embodiment 45. The method of any one of Embodiments 28 to 42, wherein the pharmaceutical composition comprises (i) buprenorphine, (ii) phosphatidylcholine, (iii) glycerol dioleate, and (iv) an organic solvent. In aspects, the organic solvent is ethanol, N-methyl-2-pyrrlidone, or a combination thereof.

Embodiment 46. The method of any one of Embodiments 28 to 42, wherein the pharmaceutical composition comprises buprenorphine, dextrose, and water.

Embodiment 47. The method of any one of Embodiments 28 to 46, wherein the pharmaceutical composition is administered prior to occurrence of opioid-induced apnea or opioid overdose.

Embodiment 48. The method of any one of Embodiments 28 to 46, further comprising treating the patient for opioid use disorder.

Embodiment 49. The method of any one of Embodiments 28 to 47, wherein the therapeutically effective amount of buprenorphine provides a sustained buprenorphine plasma concentration of about 2 ng/mL or more.

Embodiment 50. The method of any one of Embodiments 1 to 49, wherein the therapeutically effective amount of buprenorphine provides a sustained buprenorphine plasma concentration from about 2 ng/mL to about 20 ng/mL.

Embodiment 51. The method of any one of Embodiments 1 to 50, wherein the therapeutically effective amount of buprenorphine or the pharmaceutically acceptable salt thereof achieves sustained μ-opioid receptor occupancy of at least 70%.

Embodiment 52. A method of treating or preventing fentanyl-induced respiratory depression in a patient in need thereof, the method comprising administering to the patient a therapeutically effective amount of a pharmaceutical composition comprising buprenorphine, a phospholipid, a neutral diacyl lipid, and an organic solvent, to treat or prevent fentanyl-induced respiratory depression.

Embodiment 53. The method of Embodiment 52, wherein the phospholipid is phosphatidylcholine; the neutral diacyl lipid is glycerol dioleate, and the organic solvent is ethanol, N-methyl-2-pyrrolidone, or a combination thereof.

Embodiment 54. The method of Embodiment 52 or 53, wherein the therapeutically effective amount of buprenorphine is 8 mg, 16 mg, 24 mg, 32 mg, 64 mg, 96 mg, or 128 mg.

Embodiment 55. The method of any one of Embodiments 52 to 54, comprising administering the pharmaceutical composition to the patient once per week, twice per month, or once per month by subcutaneous injection.

Embodiment 56. The method of any one of Embodiments 52 to 55, wherein the pharmaceutical composition is administered prior to occurrence of fentanyl-induced respiratory depression.

Embodiment 57. The method of any one of Embodiments 52 to 56, further comprising treating the patient for opioid use disorder.

### Embodiments P1-P26

Embodiment P1. A method of treating or preventing opioid-induced respiratory depression in a patient in need thereof, the method comprising administering to the patient a therapeutically effective amount of buprenorphine or a pharmaceutically acceptable salt thereof; wherein the patient is taking an opioid; and wherein the buprenorphine or the pharmaceutically acceptable salt thereof is administered prior to occurrence of the opioid-induced respiratory depression.

Embodiment P2. A method of reducing the incidence of opioid-induced respiratory depression in a patient in need thereof, the method comprising administering to the patient a therapeutically effective amount of buprenorphine or a pharmaceutically acceptable salt thereof; wherein the patient is taking an opioid; and wherein the buprenorphine or the pharmaceutically acceptable salt thereof is administered prior to occurrence of the opioid-induced respiratory depression.

Embodiment P3. A method of treating or preventing opioid-induced apnea in a patient in need thereof, the method comprising administering to the patient a therapeutically effective amount of buprenorphine or a pharmaceutically acceptable salt thereof wherein the patient is taking an opioid; and wherein the buprenorphine or the pharmaceutically acceptable salt thereof is administered prior to occurrence of the opioid-induced apnea.

Embodiment P4. A method of reducing the incidence of opioid-induced apnea in a patient in need thereof, the method comprising administering to the patient a therapeutically effective amount of buprenorphine or a pharmaceutically acceptable salt thereof; wherein the patient is taking an opioid; and wherein the buprenorphine or the pharmaceutically acceptable salt thereof is administered prior to occurrence of the opioid-induced apnea.

Embodiment P5. A method of treating or preventing an opioid overdose in a patient in need thereof, the method comprising administering to the patient a therapeutically effective amount of buprenorphine or a pharmaceutically acceptable salt thereof; wherein the patient is taking an opioid; and wherein the buprenorphine or the pharmaceutically acceptable salt thereof is administered prior to occurrence of the opioid overdose.

Embodiment P6. A method of reducing the incidence of opioid overdose in a patient in need thereof, the method comprising administering to the patient a therapeutically effective amount of buprenorphine or a pharmaceutically acceptable salt thereof; wherein the patient is taking an opioid; and wherein the buprenorphine or the pharmaceutically acceptable salt thereof is administered prior to occurrence of the opioid overdose.

Embodiment P7. A method of treating or preventing opioid-induced respiratory depression, opioid-induced apnea, or opioid overdose in a patient being treated for pain, the method comprising administering to the patient a therapeutically effective amount of buprenorphine or a pharmaceutically acceptable salt thereof; wherein the patient is being treated for pain with an opioid.

Embodiment P8. A method of reducing the incidence of opioid-induced respiratory depression, opioid-induced apnea, or opioid overdose in a patient being treated for pain, the method comprising administering to the patient a therapeutically effective amount of buprenorphine or a pharmaceutically acceptable salt thereof; wherein the patient is being treated for pain with an opioid.

Embodiment P9. The method of any one of Embodiments P1 to P8, comprising subcutaneously administering the buprenorphine or the pharmaceutically acceptable salt thereof.

Embodiment P10. The method of any one of Embodiments P1 to P8, comprising parenterally administering the buprenorphine or the pharmaceutically acceptable salt thereof.

Embodiment P11. The method of any one of Embodiments P1 to P8, comprising intravenously administering the buprenorphine or the pharmaceutically acceptable salt thereof via intravenous injection.

Embodiment P12. The method of any one of Embodiments P1 to P8, comprising transmucosally administering the buprenorphine or the pharmaceutically acceptable salt thereof.

Embodiment P13. The method of any one of Embodiments P1 to P12, wherein the opioid is fentanyl.

Embodiment P14. The method of any one of Embodiments P1 to P12, wherein the opioid is carfentanil.

Embodiment P15. The method of any one of Embodiments P1 to P12, wherein the opioid is heroin.

Embodiment P16. The method of any one of Embodiments P1 to P12, wherein the opioid is fentanyl, carfentanil, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or a combination of two or more thereof.

Embodiment P17. The method of any one of Embodiments P1 to P16, wherein the therapeutically effective amount of buprenorphine or the pharmaceutically acceptable salt thereof achieves sustained μ-opioid receptor occupancy of at least 70%.

Embodiment P18. The method of any one of Embodiments P1 to P17, wherein the therapeutically effective amount of buprenorphine provides a sustained buprenorphine plasma concentration of about 2 ng/mL or more.

Embodiment P19. The method of any one of Embodiments P1 to P17, wherein the therapeutically effective amount of buprenorphine provides a sustained buprenorphine plasma concentration of about 2 ng/mL.

Embodiment P20. The method of any one of Embodiments P1 to P17, wherein the therapeutically effective amount of buprenorphine provides a sustained buprenorphine plasma concentration of about 5 ng/mL or more.

Embodiment P21. The method of any one of Embodiments P1 to P17, wherein the therapeutically effective amount of buprenorphine provides a sustained buprenorphine plasma concentration of about 5 ng/mL.

Embodiment P22. The method of any one of Embodiments P1 to P17, wherein the therapeutically effective amount of buprenorphine provides a sustained buprenorphine plasma concentration from about 2 ng/mL to about 15 ng/mL.

Embodiment P23. The method of any one of Embodiments P1 to P17, wherein the therapeutically effective amount of buprenorphine provides a sustained buprenorphine plasma concentration from about 2 ng/mL to about 10 ng/mL.

Embodiment P24. The method of any one of Embodiments P1 to P17, wherein the therapeutically effective amount of buprenorphine provides a sustained buprenorphine plasma concentration from about 2 ng/mL to about 8 ng/mL.

Embodiment P25. The method of any one of Embodiments P1 to P17, wherein the therapeutically effective amount of buprenorphine provides a sustained buprenorphine plasma concentration from about 2 ng/mL to about 6 ng/mL.

Embodiment P26. The method of any one of Embodiments P1 to P17, wherein the therapeutically effective amount of buprenorphine provides a sustained buprenorphine plasma concentration from about 2 ng/mL to about 5 ng/mL.

### EXAMPLES

The following examples are for purposes of illustration only and are not intended to limit the spirit or scope of the disclosure or claims.

Buprenorphine, a partial agonist at the μ-opioid receptor is used for the medication-assisted treatment of opioid use disorder. Buprenorphine has high affinity for the μ-opioid receptor and therapeutic plasma concentrations achieve at least 70% μ-opioid receptor occupancy. As a partial agonist, buprenorphine has a ceiling effect on respiratory depression such that it does not cause apnea when administered alone and minute ventilation is not suppressed beyond 50 to 60%. Buprenorphine will competitively inhibit the effects of potent, short-acting μ-opioid receptor agonists like fentanyl and carfentanil that can result in apnea and death. The objective of this trial is to determine if buprenorphine action at the μ-opioid receptor can shift the respiratory depression response to intravenous fentanyl injection to the right, thereby reducing the potential of fentanyl to cause respiratory depression, which is the usual fatal precipitant associated with intravenous fentanyl or heroin overdose.

### Example 1

The primary objectives of the Phase I Clinical Trial are to determine if buprenorphine action at the μ-opioid receptor can inhibit the respiratory depression response to intravenous fentanyl injection in healthy subjects and opioid-tolerant patients, and to determine if therapeutic concentrations achieved with administration of buprenorphine in opioid-tolerant patients protect against respiratory depression associated with high concentrations of fentanyl. More particularly, minute ventilation (L/min), respiratory rate (/min), oxygen saturation (SpO₂), tidal volume (L), end-tidal PCO₂ and end-tidal PO₂ will be measured for each breath during the baseline period and during infusion of study drugs. Peak ventilatory depression (change in minute ventilation) will be calculated based on a 1-minute average of the ventilation data of each individual subject/patient. For buprenorphine or placebo, absolute changes and percentage changes are calculated from the baseline value. For fentanyl, absolute changes and percentage changes for each bolus are calculated from the baseline value and from the pre-fentanyl baseline value immediately fentanyl bolus

The secondary objective of the clinical trial is to assess safety as determined by adverse event reporting. For Part A healthy subjects this includes: (1) the number (percentage) of subjects who experience apnea for each fentanyl dose during the placebo treatment vs. the buprenorphine treatment; and (2) the number (percentage) of subjects who require stimulation for breathing for each fentanyl dose during the placebo treatment vs. the buprenorphine treatment. For Part B opioid-tolerant subjects this includes: (1) whether the subject experiences apnea during buprenorphine treatment at the fentanyl dose, at which the subject had apnea during the placebo treatment; and (2) the fentanyl dose corresponding to the occurrence of apnea during placebo and buprenorphine infusion periods (if applicable).

The exploratory objectives of the study are: (i) to assess safety as determined by concomitant medications, laboratory test results, vital signs, physical examination findings, ECG parameters and Columbia-Suicide Severity Rating Scale (C-SSRS) responses; (ii) to evaluate pharmacokinetics of buprenorphine during primed-continuous intravenous infusion; (iii) to evaluate pharmacokinetics of fentanyl with repeated intravenous bolus injections; (iv) to explore changes in ventilation parameters during the intravenous administration of buprenorphine and fentanyl; and (v) to build a model describing the pharmacokinetic (PK)/pharmacodynamic (PD) interaction between buprenorphine and fentanyl concentrations and their effect on ventilatory parameters.

More particularly, the safety and tolerability endpoints are to assess safety as determined by reporting of treatment-emergent (serious) adverse events ((S)AEs), concomitant medications, laboratory test results, vital signs, ECG parameters, physical examination findings, and Columbia-Suicide Severity Rating Scale (C-SSRS) responses.

The pharmacokinetic endpoints will be determined for buprenorphine following each treatment and will be derived by non-compartmental analysis of the following plasma concentration-time data: (i) the maximum plasma concentration at the end of the bolus (Cmax); (ii) the area under the plasma concentration-time curve from zero to t of the last measured concentration above the limit of quantification (AUC0-last); and (iii) the average concentration during the fentanyl dose escalation Cavg (2-6h), which is calculated as AUC(2-6h)/4h.

The pharmacokinetic endpoints will be determined for fentanyl following each bolus and will be derived by non-compartmental analysis of the following plasma concentration-time data: (i) the area under the plasma concentration-time curve for each dosing interval (AUC0-tau); (ii) the maximum plasma concentration for each dosing interval (Cmax); (iii) the time to reach maximum plasma concentration for each dosing interval (tmax); and (iv) other parameters, including apparent volume of distribution (Vz/F), apparent clearance (CL/F), and other parameters as appropriate, as well as dose adjusted parameters, may be determined.

The pharmacodynamic endpoints, including minute ventilation (L/min), respiratory rate (/min), tidal volume (L), oxygen saturation (SpO₂), end-tidal PCO₂ and end-tidal PO₂ will be measured for each breath during the baseline period and during infusion of study drugs. The following parameters will be calculated: (i) peak changes in other ventilation parameters will be calculated for buprenorphine or placebo, absolute changes and percentage changes are calculated from the baseline value. For fentanyl, absolute changes and percentage changes for each bolus are calculated from the baseline value and from the pre-fentanyl baseline value immediately before the first fentanyl bolus; (ii) area under the curve in ventilation parameters will be calculated based on a 1-minute average of the ventilation data of each individual subject/patient. For buprenorphine or placebo, changes are calculated from the baseline value. For fentanyl, changes are calculated for each bolus are calculated from the baseline value and from the pre-fentanyl baseline value immediately before the first fentanyl bolus; (iii) when possible, time to peak effect (min) and time to end of effect (i.e., return to baseline in minutes) will be calculated for each for the initial buprenorphine/placebo period and each fentanyl bolus; (iv) EC50 and Emax for buprenorphine and fentanyl effects on minute ventilation as determined by PK/PD models; and (v) Sedation Visual Analogue Scale (VAS) administered before the first fentanyl bolus and at the conclusion of each bolus period.

Part A is a 3-period study in approximately 18 healthy subjects. A minimum of 5 subjects of each sex will be included to address any potential differences between sexes. The first 2 periods (Period 1 and Period 2) have a single-blind, placebo-controlled, cross-over design, where subjects will be randomized in a 1 to 1 ratio to 2 treatment sequences determined by the order in which they receive buprenorphine or matching placebo. Period 3 is an open-label design, where the same subjects will receive buprenorphine only. Period 3 is optional, and not all subjects are required to participate; the sponsor and Investigator collectively determine if Period 3 is required for each subject. In total, about 6 subjects will participate in a third investigational period. Per 3 subjects, the buprenorphine dose will be determined based on safety and pharmacodynamic results, leading to approximately 6 dose cohorts and a minimum of 3 subjects assigned to any buprenorphine dose level. Every effort will be made to represent both sexes in each buprenorphine dose level. Healthy subjects' participation is a maximum of 13 weeks.

Part A inclusion criteria for healthy subjects includes: (1) signed the informed consent form (ICF) and able to comply with the study requirements and restrictions listed therein; (2) male and female subjects, age 18 to 45 years, inclusive; (3) women of childbearing potential (defined as all women who are not surgically sterile or postmenopausal for at least 1 year prior to informed consent) must have a negative serum pregnancy test prior to enrollment and must agree to use a medically acceptable means of contraception from screening through at least 3 months after the last dose of study drug; (4) body mass index (BMI) of 18 to 30 kg/m², inclusive; (5) healthy as defined by the Investigator, based on a medical evaluation that includes the subject's medical and surgical history, physical examination, vital signs, 12-lead electrocardiogram (ECG), hematology, blood chemistry, and urinalysis; (6) no history of substance use disorder; and (7) no current use of any central nervous system (CNS) depressants prescribed or otherwise.

Part B is an open label study in approximately 8 opioid-tolerant patients. All opioid-tolerant patients will receive placebo plus fentanyl during Period 1 and buprenorphine plus fentanyl during Period 2. A minimum of 3 subjects of each sex will be included in the population in order to address any potential differences due to sex. opioid-tolerant patients' participation is about 8 weeks.

Part B inclusion criteria for opioid-tolerant patients includes: (1) signed the ICF and able to comply with the requirements and restrictions listed therein; (2) male and female, age 18 to 55 years, inclusive; (3) women of childbearing potential (defined as all women who are not surgically sterile or postmenopausal for at least 1 year prior to informed consent) must have a negative serum pregnancy test prior to enrollment and must agree to use a medically acceptable means of contraception from screening through at least 3 months after the last dose of study drug; (4) BMI of 18 to 32 kg/m², inclusive; (5) Opioid-tolerant patients administered opioids at daily doses 90 mg oral morphine equivalents; (6) stable as defined by the Investigator, based on a medical evaluation that includes the patient's medical and surgical history, physical examination, vital signs, 12-lead ECG, hematology, blood chemistry, and urinalysis; (7) no current use of any CNS depressants, besides opioids, prescribed or otherwise for 5 half-lives of the product before first study drug administration.

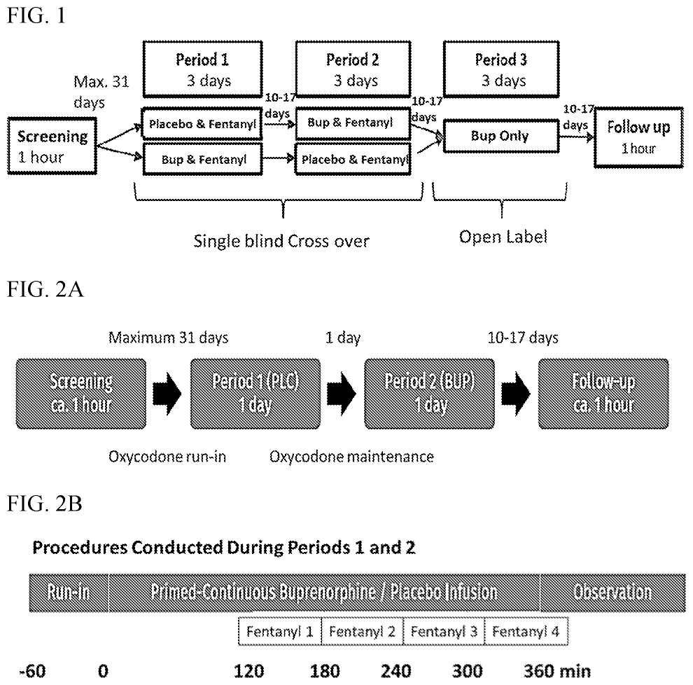

All healthy subjects (Part A) will be screened up to 31 days prior to study drug administration. Subjects who sign informed consent and meet all entry criteria may be enrolled. All healthy subjects in Part A will be studied in 2 or 3 periods, with 10-17 days between the periods. All subjects will receive ondansetron before dosing with buprenorphine or placebo in order to minimize the nausea effect of opioids. Subjects will receive the same doses of fentanyl challenges in Periods 1 and 2; however, fentanyl will not be dosed in Period 3. Buprenorphine doses will be set per dosing cohort. All subjects will be admitted the evening before each study period. After study periods are completed, subjects will be transferred to the Post-Anesthesia Care Unit (PACU) for overnight monitoring. The overview of the study design for healthy subjects is shown in FIG. 1.

All opioid-tolerant patients (Part B) will be screened up to 31 days prior to study drug administration. Subjects who sign informed consent and meet all entry criteria may be enrolled. All opioid-tolerant patients in Part B will be studied in 2 separate periods, with at least 40 hours washout in between, while returning between the two periods. All opioid-tolerant patients will be transitioned to oral oxycodone before Period 1, and they will be admitted to 2-5 days before Period 1 in order to ensure washout of the patients' usual opioids and a stable dose with an adequate bridging schedule has been reached. Tolerance to opioid effects is poorly characterized in subjects receiving long-term opioids; therefore, opioid-tolerant patients in Part B will receive placebo plus fentanyl challenges in Period 1 in order to optimize the fentanyl dose escalation before buprenorphine and fentanyl are co-administered in Period 2. Due to the short half-life of fentanyl, opioid-tolerant patients will return and will continue onto Period 2 after a washout of at least 40 hours. Opioid-tolerant patients will be escorted to the treatment center on the morning of Day 1 and Day 3. In the event that a washout period significantly longer than 40 hours is required between Periods 1 and 2, opioid-tolerant patients may stay for a longer period until Period 2 begins. The overview of the study design for opioid-tolerant patients is shown in FIG. 2.

The End of Study/Early Termination visit will be completed 10-17 days after the final study period dosing.

To study ventilation, the dynamic end-tidal forcing technique will be used, as described by Dahan et al, Br J Anaesth., 94:825-834, (2005), and Yassen et al, Clin Pharmacol Ther., 81:50-58 (2007). This technique enables the Investigator to force end-tidal PCO₂ and end-tidal PO₂ to follow a specific pattern in time. End-tidal PCO₂ and PO₂ will be clamped to about 7 kPa and 14.5 kPa, respectively, until minute ventilation (VE) reaches 20 to 24 L/min. Subjects breathe through a face mask and receive fresh gas (45 L/min) with oxygen, carbon dioxide and nitrogen adjusted to obtain the desired end-tidal concentrations. The inspired and expired gas flows are measured using a pneumotachograph and the oxygen and carbon dioxide concentrations are measured using a gas monitor; a pulse oximeter continuously measures the oxygen saturation of arterial hemoglobin with a finger probe. All relevant variables are available for online analysis and stored on a breath-to-breath basis for further analysis.

After baseline ventilation stabilizes (30 to 45 minutes), subjects (all healthy subjects and opioid-tolerant patients as needed) will receive ondansetron 4 mg intravenous and a primed-continuous intravenous buprenorphine (or placebo) infusion will be initiated at doses expected to achieve target concentrations resulting in approximately 25% to 50% suppression of baseline minute ventilation. Buprenorphine infusion will continue for 6 hours (360 minutes) and fentanyl boluses will be administered at 120, 180, 240 and 300 minutes (in Periods 1 and 2) to complete a 4-step intravenous bolus dose escalation. Study drug administration concludes at 360 minutes, and study subjects will be monitored for a minimum of 3 hours before transfer to the PACU. If a subject indicates that he or she wants to discontinue the experiment or in case of an adverse event, all infusions will be discontinued for that period. Subjects with a procedure-related adverse event will be treated according to established ventilatory support and opioid reversal protocols. A procedure-related adverse event is defined by loss of respiratory activity for 60 seconds or longer, despite active stimulation of the subject; end-tidal partial pressure of carbon dioxide greater than 67.5 mmHg, O₂ saturation less than 85% for at least 2 minutes, or any other situation or condition that may interfere with the health of the participant. If an Investigator stimulates a subject to breathe or gives supplemental oxygen as needed to prevent an adverse event, the subject will not proceed to the next fentanyl dose and the period will terminate early.

The investigational drugs used in the study will be 0.3 mg/ml buprenorphine intravenous injection (TEMGESIC® injection, Indivior); 0.05 mg/mL fentanyl intravenous injection; 0.9% normal saline placebo intravenous injection. The non-investigational drugs used in the study will be 2 mg/mL ondansetron intravenous injection, and oxycodone tablets (for Part B only).

All healthy subjects will be dosed with 4 mg ondansetron at least 15 minutes before dosing with buprenorphine or placebo. Buprenorphine dosing is flexible and infusion rates will be selected to target approximately 25 to 50% respiratory depression. The potential buprenorphine continuous infusion rates, based on published reports providing a dose-response for buprenorphine effects on analgesia and ventilation, include 0.005, 0.01, 0.02, 0.05, 0.1 mg and 0.2 mg/70 kg/h. See Dahan et al, Br J Anaesth., 94:825-834, (2005); Yassen et al, Clin Pharmacol Ther., 81:50-58 (2007); Dahan et al, Br J Anaesth., 96:627-632 (2006). The initial dose cohort of 3 subjects will receive the starting dose of buprenorphine listed below and subsequent doses will be selected to explore the full range of effects on ventilation. The starting dose of buprenorphine is expected to yield significant receptor occupancy and produce differential effects on respiratory depression relative to placebo. Doses for subsequent dose cohorts will be selected from the potential doses listed based on subject tolerability and respiratory effects from earlier dose cohorts. Starting buprenorphine dose: target concentration 0.5 ng/mL: (a) 0.125 mg/70 kg bolus, (b) 0.05 mg/70 kg/h for 360 minutes. Subjects will be dosed with 90-second bolus injections of fentanyl using the following escalation: (a) bolus 1: 0.075 mg/70 kg; (b) bolus 2: 0.15 mg/70 kg (hold if apnea observed in earlier steps); (c) bolus 3: 0.25 mg/70 kg (hold if apnea observed in earlier steps); and (d) bolus 4: 0.35 mg/70 kg (hold if apnea observed in earlier steps).

Opioid-tolerant patients in Part B will undergo a washout of their own opioids during which time these will be replaced by oral oxycodone; they will continue to receive stable doses of oxycodone from 2-5 days before Period 1 until discharge at the end of Period 2. Not less than 15 hours prior to the start of each dose administration of buprenorphine/placebo, the last oxycodone dose will be administered. Placebo and fentanyl will be administered during Period 1 to permit dose escalation to full respiratory effects of fentanyl before assessing the interaction with buprenorphine during the second study period. The low and high doses of buprenorphine listed below have been shown to achieve 50% and >80% receptor occupancy measured by positron emission tomography with [¹¹C]carfentanil radioligand. See Greenwald et al, Neuropsychopharmacology, 28:2000-2009 (2003); Greenwald et al, Drug Alcohol Depend., 144:1-11 (2014). Buprenorphine doses can be adjusted as needed up to a maximum infusion rate of 0.75 mg/70 kg/h based on experimental observations in Part A. Buprenorphine low dose target concentration of 1.0 ng/mL to be reached by: (i) 0.25 mg/70 kg bolus; and (ii) 0.1 mg/70 kg/h for 360 minutes. Buprenorphine high dose target concentration of 5.0 ng/mL to be reached by: (i) 1.25 mg/70 kg bolus; and (ii) 0.5 mg/70 kg/h for 360 minutes. Because chronic opioid administration via prescription opioids can elicit marked tolerance to opioid effects, fentanyl bolus dose escalation will be performed on an individual basis. If the initial fentanyl boluses have no visible impact on ventilation, then the escalation will be pushed beyond the doses listed below. The maximum proposed dose is 2.0 mg/70 kg depending on observations during fentanyl dose escalation. If minute ventilation does not fall below 2/3 of baseline at the maximum dose of fentanyl, then this patient will not be continued into the second period and will be replaced. The proposed fentanyl administration is: (i) bolus 1: 0.25 mg/70 kg; (ii) bolus 2: 0.35 mg/70 kg (hold if apnea observed in earlier steps); (iii) bolus 3: 0.5 mg/70 kg (hold if apnea observed in earlier steps); and (iv) bolus 4: 0.7 mg/70 kg (hold if apnea observed in earlier steps).

The study will demonstrate that buprenorphine reduces respiratory depression and/or reverses respiratory depression caused by fentanyl in healthy subjects and in opioid-tolerant subjects. The results will demonstrate that buprenorphine can be used to treat opioid-induced respiratory depression in patients. In addition, the results will also demonstrate: (i) that buprenorphine can be used in conjunction with other opioids (e.g., fentanyl) to treat pain in order to reduce the risk that the patient will experience respiratory depression caused by the other opioids (e.g., fentanyl); (ii) that buprenorphine can be used in conjunction with other opioids (e.g., fentanyl) to treat pain in order to reduce the risk that the patient will experience apnea caused by the other opioids (e.g., fentanyl); (iii) that buprenorphine can be used in conjunction with other opioids (e.g., fentanyl) to treat pain in order to reduce the risk that the patient will experience an overdose caused by the other opioids (e.g., fentanyl); (iv) buprenorphine can be used to prevent opioid-induced respiratory depression in patients; (v) buprenorphine can be used to reduce the incidence of opioid-induced respiratory depression in patients; (vi) buprenorphine can be used to treat opioid-induced apnea in patients; (vii) buprenorphine can be used to prevent opioid-induced apnea in patients; (viii) buprenorphine can be used to reduce the incidence of opioid-induced apnea in patients; (ix) buprenorphine can be used to treat opioid overdose in patients; (x) buprenorphine can be used to prevent opioid overdose in patients; (xi) buprenorphine can be used to reduce the incidence of opioid overdose in patients; or (xii) a combination of two or more of the foregoing.

### Example 2

Eight opioid-tolerant patients using more than 90 mg daily morphine equivalents completed this open label, two-period crossover study. The patient demographics are shown in Table 1.

TABLE 1

| Buprenorphine Dose | Patient ID | Sex | Age | BMI | Drug Usage |
|---|---|---|---|---|---|
| 1 ng/mL | 201 | F | 44 | 23.6 | Oxycodone 60 ng/day |
| 1 ng/mL | 205 | M | 46 | 29.6 | Fentanyl patch 25 mcg/hour |
| | | | | | Oxycodone 60 mg/day |
| | | | | | Marijuana |
| 2 ng/mL | 206 | F | 33 | 30.8 | Fentanyl patch 75 mcg/hour |
| | | | | | Oxycodone 90 mg/day |
| | | | | | Tapentadol 50 mg/day |
| 2 ng/mL | 208 | M | 43 | 22.0 | Buprenorphine 16 mg/day |
| | | | | | Cocaine |
| | | | | | Marijuana |
| 2 ng/mL | 1207 | F | 31 | 23.2 | Oxycodone 60 mg/day |
| | | | | | Marijuana |
| 5 ng/mL | 202 | M | 52 | 25.1 | Heroin 250 mg/day (smoke) |
| | | | | | Cocaine |
| | | | | | Marijuana |
| 5 ng/mL | 203 | F | 52 | 31.5 | Fentanyl patch 50 mcg/hour |
| 5 ng/mL | 204 | F | 34 | 21.0 | Fentanyl patch 25 mcg/hour |
| | | | | | Oxycodone 60 mg/day |
| | | | | | Marijuana |

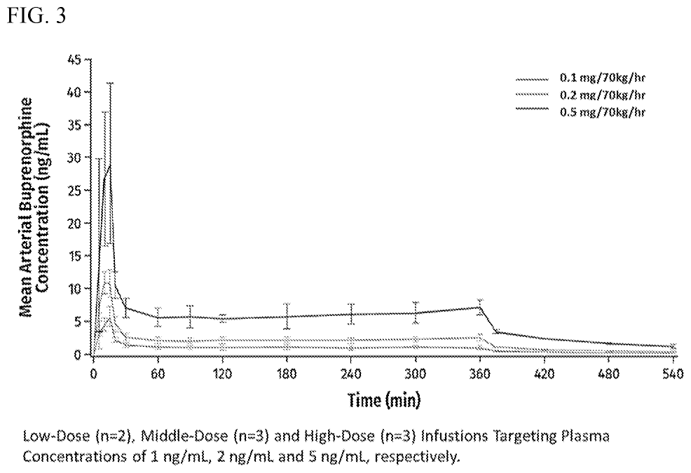

The trial lasted about 8 weeks, including screening, Period 1, Period 2, and end of study follow-up (FIG. 2). As shown in FIG. 3, participants received placebo, fentanyl during Period 1 (Day 1) and buprenorphine, and fentanyl during Period 2 (Day 3). Minute ventilation (MV) was measured by the dynamic end-tidal forcing technique, as described by Yassen et al, Clin Pharmacol Therapeut, 81:50-8 (2007). End-tidal PCO₂ and PO₂ were clamped at approximately 7 and 14.5 kPa, respectively, until MV (tidal volume x respiratory rate) reached 20 to 24 L/min. Participants breathed through a face mask and received fresh gas with O₂, CO₂, and N₂ adjusted to obtain the desired end-tidal concentrations. The inspired and expired gas flows were measured using a pneumotachograph, and the O₂ and CO₂ concentrations were measured using a gas monitor, a pulse oximeter continuously measured the oxygen saturation. Drug effects were measured as a decrease in MV, number/duration of apnoeic events lasting more than 20 seconds, the need for ventilatory stimulation, and changes in oxygen saturation.

Once ventilation was stable, participants received ondansetron 4 mg IV, and a primed-continuous IV buprenorphine (or placebo) infusion was initiated. Buprenorphine infusion targeted plasma concentrations of 1 ng/mL (low dose), 2 ng/mL (middle dose), and 5 ng/mL (high dose), consistent with levels achieved with the FDA-approved doses of SUBLOCADE® (buprenorphine injection, Indivior). Buprenorphine infusion continued for 360 minutes and fentanyl boluses were administered at 120, 180, 240, and 300 minutes to complete a 4-step IV bolus dose escalation shown in Table 2. Fentanyl dose escalation was discontinued at the investigator's discretion if participants experienced apnoea, required ventilatory stimulation, or had an unstable breathing pattern.

TABLE 2

| | Buprenorphine Dosing | | Fentanyl Dosing | |
|---|---|---|---|---|
| | Prime | Continuous | | Bolus |
| | (mg/70 kg) | (mg/70 kg/h) | | (mg/70 kg) |
| 1 ng/mL | 0.25 | 0.10 | Fentanyl Dose 1 | 0.25 |
| Buprenorphine Dose | | | | |
| 2 ng/mL | 0.50 | 0.20 | Fentanyl Dose 2 | 0.35 |
| Buprenorphine Dose | | | | |
| 5 ng/mL | 1.25 | 0.30 | Fentanyl Dose 3 | 0.50 |
| Buprenorphine Dose | | | | |
| | | | Fentanyl Dose 4 | 0.70 |

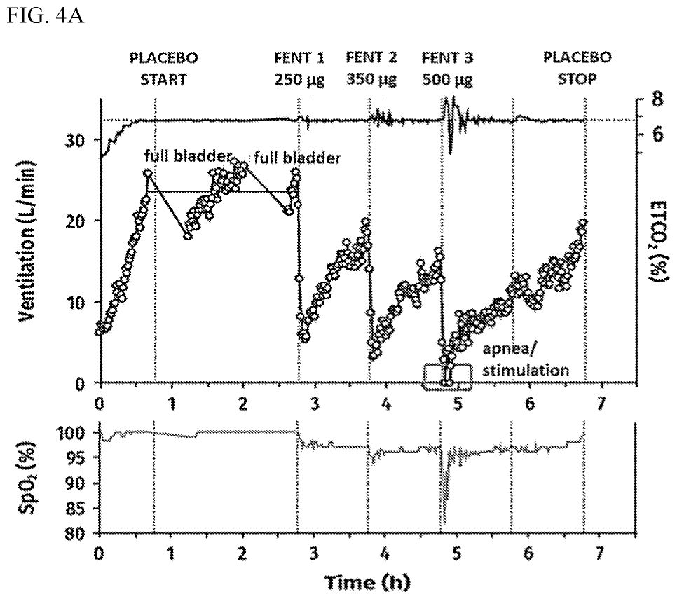

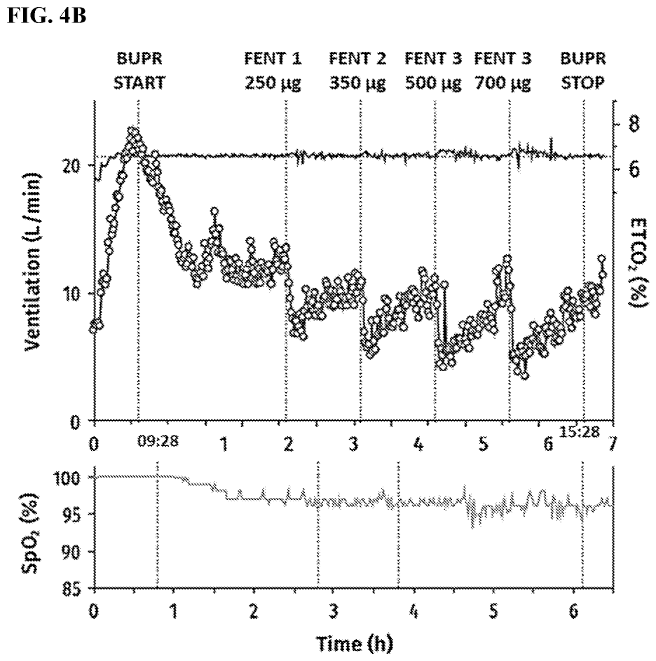

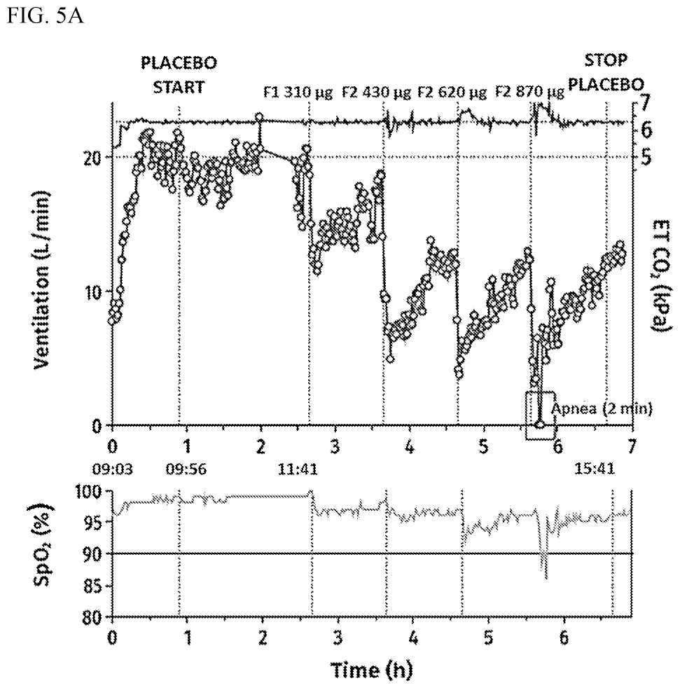

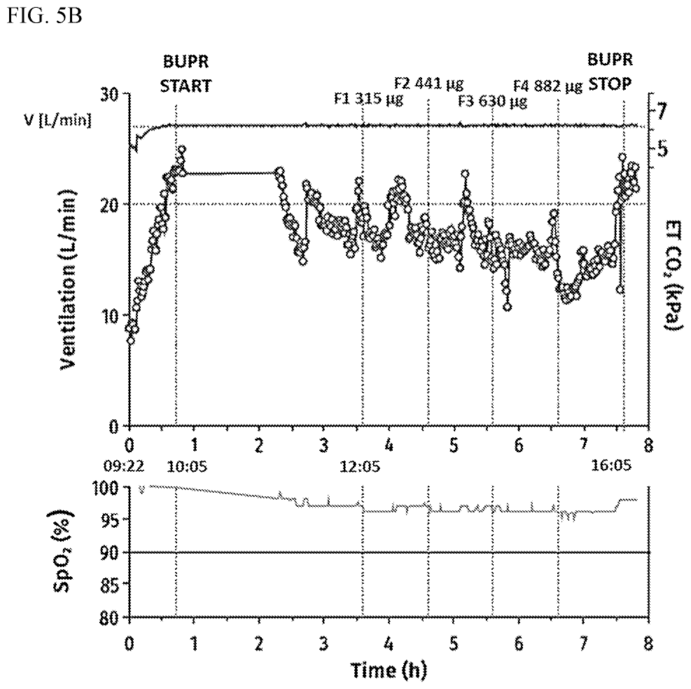

During the PLC period, abrupt declines in MV were generally evident following each fentanyl bolus and 6 of 8 patients experienced one or more apneic events requiring verbal ventilatory stimulation to maintain adequate MV. IV fentanyl dose escalation was stopped early after the second (n=2) or third bolus (n=3) in 5 subjects because of prolonged apnea or changes in oxygen saturation (5 subjects had oxygen saturation values <90%). During the BUP period, each patient completed four fentanyl boluses and only 1 patient experienced an apneic episode after the third and fourth boluses. With BUP, none of the patients required verbal ventilatory stimulation and oxygen saturation did not drop below 90%. For the low dose BUP infusion targeting 1 ng/mL, declines in MV were evident after fentanyl boluses and the one patient with apneas during BUP infusion was in this group. For the high dose BUP infusion targeting 5 ng/mL, marked changes in MV did not occur after the fentanyl infusions and repeated apneic events did not occur. The results for end-tidal CO2 and minute ventilation and oxygen saturation (SpO2) for the first patient to receive injections of buprenorphine and fentanyl boluses are shown in FIGS. 4A-4B, FIGS. 5A-5B, and FIGS. 6A-6B. These results are also summarized in Table 3.

TABLE 3

| | Placebo or | No. Fentanyl | |
|---|---|---|---|
| Subject | Buprenorphine Dose | Boluses | Notes |
| 201 | Placebo | 3 | Apnoea after 3rd bolus. Intermittent for 5 |
| | | | minutes with stimulations. ↓O₂ saturation. |
| | 1 ng/mL | 4 | No apnoea events |
| 205 | Placebo | 2 | Prolonged apnoea after 2nd bolus. Lasted |
| | | | nearly 10 minutes with stimulation required. |
| | | | ↓O₂ saturation. |
| | 1 ng/mL | 4 | Apnoea after 3rd bolus. No stimulation. |
| | | | Intermittent apnoea after 4th bolus but no |
| | | | stimulation required. ↓O₂ saturation. |
| 206 | Placebo | 4 | Apnoea after 4th bolus for 2 minutes with |
| | | | stimulation required. ↓O₂ saturation. |
| | 2 ng/mL | 4 | No apnoea events |
| 208 | Placebo | 4 | Prolonged apnoea after 4th bolus. Lasted 12 |
| | | | minutes with stimulation required. ↓O₂ |
| | | | saturation. |
| | 2 ng/mL | 4 | No apnoea events |
| 1207 | Placebo | 4 | No apnoea events |
| | 2 ng/mL | 4 | No apnoea events |
| 202 | Placebo | 4 | Prolonged apnoea after 4th bolus. Lasted 25 |
| | | | minutes with stimulation required. ↓O₂ |
| | | | saturation. |
| | 5 ng/mL | 4 | No apnoea events |
| 203 | Placebo | 2 | Apnoea after 2nd bolus. 2 events with verbal |
| | | | stimulation. |
| | 5 ng/mL | 4 | Brief apnoea after 2nd bolus but not others. |
| | | | No stimulation. |
| 204 | Placebo | 3 | Apnoea after 3rd bolus. Intermittent for 5 |
| | | | minutes with unstable breathing pattern. |
| | 5 ng/mL | 4 | No apnoea events |

For the placebo sessions, abrupt declines in MV were generally evident following each fentanyl bolus (FIG. 4A, 5A, 6A); six of eight participants experience 1 or more apnoeic events requiring verbal ventilatory stimulation; IV fentanyl dose escalation was stopped early after the 2nd bolus (n=2) or 3rd bolus (n=2) in four participants because of prolonged apnoea or changes in oxygen saturation; and 5 participants had oxygen saturation values less than 90%.

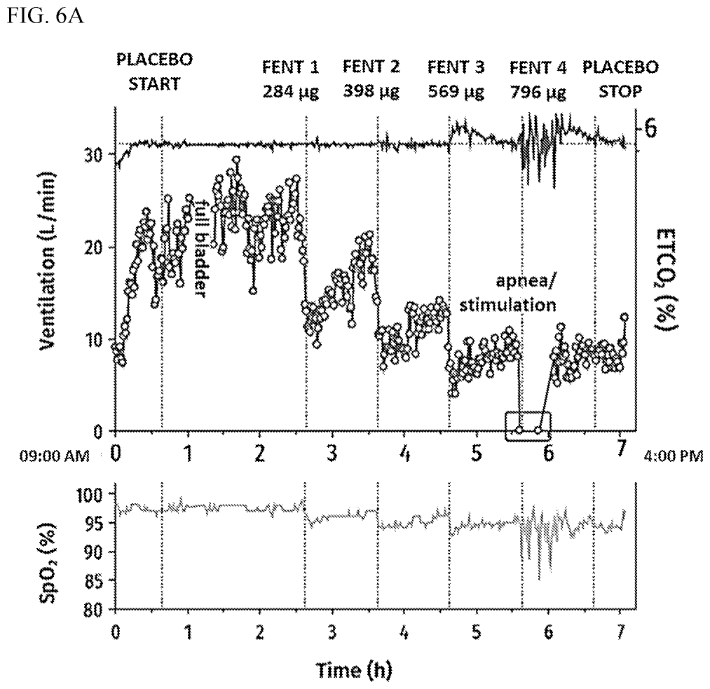

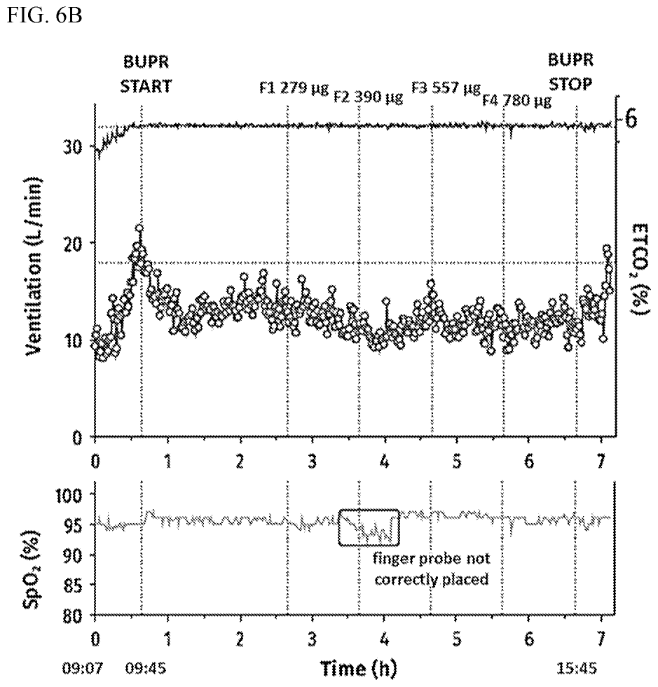

For the buprenorphine sessions, each participant completed all four fentanyl boluses; only 1 participant experienced an apnoeic episode after the 3rd or 4th boluses; verbal ventilatory stimulation was not required; and oxygen saturation did not drop below 90%. For the 1 ng/mL buprenorphine dose response, declines in MV were evident after fentanyl boluses (FIG. 4B), and the one participant with the apnoeic event during buprenorphine infusion was in this group. For the 5 ng/mL buprenorphine dose response, marked changes in MV did not occur after the fentanyl infusions and repeated apnoeic events did not occur (FIG. 6B).

Buprenorphine acts as a competitive inhibitor of fentanyl boluses up to 700 mcg/70 kg. This competitive inhibition reduces the magnitude of the fentanyl respiratory depression, most notable at buprenorphine concentrations greater than or equal to 2 ng/mL and 5 ng/mL. The study shows that patients can be protected against fentanyl-induced respiratory depression through the administration of greater than or equal to 2 ng/mL buprenorphine sustained plasma concentrations and 5 ng/mL buprenorphine sustained plasma concentrations.

While various embodiments and aspects are shown and described herein, it will be clear to the skilled artisan that such embodiments and aspects are provided by way of example. Variations, changes, and substitutions will occur to the skilled artisan. It will be understood that various alternatives to the embodiments described herein can be used.

1. A method of treating or preventing fentanyl-induced respiratory depression in a patient in need thereof, the method comprising administering to the patient a therapeutically effective amount of a pharmaceutical composition comprising buprenorphine free base, a poly(lactide-co-glycolide) copolymer, and N-methyl-2-pyrrolidone, to treat or prevent fentanyl-induced respiratory depression.

2. The method of claim 1, further comprising: (a) administering the pharmaceutical composition to the patient once per month by subcutaneous injection; (b) administering the pharmaceutical composition prior to occurrence of fentanyl-induced respiratory depression; and/or (c) treating the patient for opioid use disorder.

3. The method of claim 1, wherein the pharmaceutical composition comprises: (a) about 18 wt % buprenorphine free base, about 32 wt % of a 50:50 poly(DL-lactide-co-glycolide) copolymer, and about 50 wt % of N-methyl-2-pyrrolidone; and/or (b) about 100 mg of buprenorphine free base or about 300 mg of buprenorphine free base.

4-6. (canceled)

7. A method of treating or preventing opioid-induced respiratory depression or reducing the incidence of opioid-induced respiratory depression in a patient in need thereof, the method comprising administering to the patient a therapeutically effective amount of a pharmaceutical composition comprising buprenorphine or a pharmaceutically acceptable salt thereof.

8. The method of claim 7, for: (a) treating or preventing opioid-induced respiratory depression; and/or (b) reducing the incidence of opioid-induced respiratory depression.

9. (canceled)

10. The method of claim 7, wherein the opioid is: (a) fentanyl, a fentanyl analogue, carfentanil, a carfentanil analogue, heroin, butorphanol, oxycodone, hydrocodone, hydromorphone, levorphanol, oxymorphone, opium, meperidine, morphine, codeine, dihydrocodeine, methadone, pentazocine, tapentadol, tramadol, heroin, or a combination of two or more thereof; (b) acetylfentanyl, butyrfentanyl, para-tolylfentanyl, 3-methylfentanyl, α-methylfentanyl, mefentanyl, phenaridine, ohmefentanyl, or mirfentanil; (c) sufentanil, remifentanil, alfentanil, lofentanil, brifentanil, or trefentanil; (d) fentanyl; and/or (e) carfentanil.

11-14. (canceled)

15. The method of claim 7, comprising: (a) administering the pharmaceutical composition to the patient once per week by subcutaneous injection; (b) administering the pharmaceutical composition to the patient twice per month by subcutaneous injection; (c) administering the pharmaceutical composition to the patient once per month by subcutaneous injection; (d) administering the pharmaceutical composition to the patient once every two months by subcutaneous injection; and/or (e) parenterally administering the pharmaceutical composition to the patient.

16-19. (canceled)

20. The method of claim 7, wherein: (a) the pharmaceutical composition is a long-acting pharmaceutical composition; (b) the pharmaceutical composition comprises buprenorphine, a poly(lactide-co-glycolide) copolymer, and N-methyl-2-pyrrolidone; (c) the pharmaceutical composition comprises about 18 wt % buprenorphine free base, about 32 wt % of a 50:50 poly(DL-lactide-co-glycolide) copolymer, and about 50 wt % of N-methyl-2-pyrrolidone; (d) the pharmaceutical composition comprises (i) buprenorphine, (ii) phosphatidylcholine, (iii) glycerol dioleate, and (iv) ethanol, N-methyl-2-pyrrlidone, or a combination thereof; and/or (e) the pharmaceutical composition comprises buprenorphine, dextrose, and water.

21-24. (canceled)

25. The method of claim 7, wherein: (a) the therapeutically effective amount of buprenorphine provides a sustained buprenorphine plasma concentration of about 2 ng/mL or more; (b) the pharmaceutical composition is administered to the patient prior to occurrence of opioid-induced respiratory depression; and/or (c) the method further comprises treating the patient for opioid use disorder.

26-27. (canceled)

28. A method of treating or preventing opioid-induced apnea, reducing the incidence of opioid-induced apnea, treating or preventing an opioid overdose, or reducing the incidence of opioid overdose in a patient in need thereof, the method comprising administering to the patient a therapeutically effective amount of a pharmaceutical composition comprising buprenorphine or a pharmaceutically acceptable salt thereof.

29. The method of claim 28, for: (a) treating or preventing opioid-induced apnea; (b) reducing the incidence of opioid-induced apnea; (c) treating or preventing an opioid overdose; and/or (d) reducing the incidence of opioid overdose.

30-32. (canceled)

33. The method of claim 28, wherein the opioid is: (a) heroin, butorphanol, oxycodone, hydrocodone, hydromorphone, levorphanol, oxymorphone, opium, meperidine, morphine, codeine, dihydrocodeine, methadone, pentazocine, tapentadol, tramadol, or a combination of two or more thereof; (b) fentanyl, acetylfentanyl, butyrfentanyl, para-tolylfentanyl, 3-methylfentanyl, α-methylfentanyl, mefentanyl, phenaridine, ohmefentanyl, or mirfentanil; (c) fentanyl; (d) sufentanil, remifentanil, alfentanil, lofentanil, brifentanil, or trefentanil; and/or (e) carfentanil.

34-37. (canceled)

38. The method of claim 28, comprising: (a) administering the pharmaceutical composition to the patient once per week by subcutaneous injection; (b) The method of claim 28, comprising administering the pharmaceutical composition to the patient once per month by subcutaneous injection; (c) The method of claim 28, comprising administering the pharmaceutical composition to the patient once every two months by subcutaneous injection; (d) The method of claim 28, comprising parenterally administering the pharmaceutical composition to the patient.

39-41. (canceled)

42. The method of claim 28, wherein the pharmaceutical composition: (a) is a long-acting pharmaceutical composition; (b) The method of claim 28, wherein the pharmaceutical composition comprises buprenorphine, a poly(lactide-co-glycolide) copolymer, and N-methyl-2-pyrrolidone; (c) The method of claim 43, wherein the pharmaceutical composition comprises about 18 wt % buprenorphine free base, about 32 wt % of a 50:50 poly(DL-lactide-co-glycolide) copolymer, and about 50 wt % of N-methyl-2-pyrrolidone; (d) The method of claim 28, wherein the pharmaceutical composition comprises (i) buprenorphine, (ii) phosphatidylcholine, (iii) glycerol dioleate, and (iv) ethanol, N-methyl-2-pyrrlidone, or a combination thereof; (e) The method of claim 28, wherein the pharmaceutical composition comprises buprenorphine, dextrose, and water; and/or (f) The method of claim 28, wherein the pharmaceutical composition comprises buprenorphine, dextrose, and water.

43-46. (canceled)

47. The method of claim 28, wherein: (a) the pharmaceutical composition is administered prior to occurrence of opioid-induced apnea or opioid overdose; (b) the method further comprises treating the patient for opioid use disorder; (c) the therapeutically effective amount of buprenorphine provides a sustained buprenorphine plasma concentration of about 2 ng/mL or more; (d) the therapeutically effective amount of buprenorphine provides a sustained buprenorphine plasma concentration from about 2 ng/mL to about 20 ng/mL; and/or (e) the therapeutically effective amount of buprenorphine or the pharmaceutically acceptable salt thereof achieves sustained μ-opioid receptor occupancy of at least 70%.

48-51. (canceled)

52. A method of treating or preventing fentanyl-induced respiratory depression in a patient in need thereof, the method comprising administering to the patient a therapeutically effective amount of a pharmaceutical composition comprising buprenorphine, a phospholipid, a neutral diacyl lipid, and an organic solvent, to treat or prevent fentanyl-induced respiratory depression.

53. The method of claim 52, wherein: (a) (i) the phospholipid is phosphatidylcholine, (ii) the neutral diacyl lipid is glycerol dioleate, and (iii) the organic solvent is ethanol, N-methyl-2-pyrrolidone, or a combination thereof; and/or (b) the therapeutically effective amount of buprenorphine is 8 mg, 16 mg, 24 mg, 32 mg, 64 mg, 96 mg, or 128 mg.

54. (canceled)

55. The method of claim 52, comprising: (a) administering the pharmaceutical composition to the patient once per week, twice per month, or once per month by subcutaneous injection; (b) administering the pharmaceutical composition prior to occurrence of fentanyl-induced respiratory depression; and/or (c) treating the patient for opioid use disorder.

56-57. (canceled)
